(19) United States
(12) Patent Application Publication
(10) Pub. No.: US 2003/0199073 A1
(43) Pub. Date: Oct. 23, 2003

(54) Production of recombinant human lysosomal alpha-mannosidase

(75) Inventors: Jens Fogh, Lynge (DK); Meher Irani, Seattle, WA (US); Claes Andersson, Taby (SE); Cecilia Weigelt, Stockholm (SE)

Correspondence Address: BROWDY AND NEIMARK, P.L.L.C., 624 NINTH STREET, NW, SUITE 300, WASHINGTON, DC, 20001-5303, US

(73) Assignee: HemeBiotech A/S, Hillerod (DK)

(21) Appl. No.: 10/315,150

(22) Filed: Dec. 10, 2002

Related U.S. Application Data
(63) Continuation-in-part of application No. PCT/DK02/00388, filed on Jun. 7, 2002.
(60) Provisional application No. 60/296,142, filed on Jun. 7, 2001.

(51) Int. Cl. C12Q 1/68; A61K 38/47; C12N 9/24; C12P 21/02; C12N 5/06

(52) U.S. Cl. 435/200; 435/69.1; 435/320.1; 435/325; 435/6; 424/94.61

(57) ABSTRACT

The present invention relates to a cell capable of producing recombinant human LAMAN (rhLAMAN), said cell comprising the 3066 basepair EcoRI-XbaI fragment of a human cDNA which codes for a human LAMAN protein in which the position corresponding to position 186 of the full length hLAMAN protein is Aspartic acid (Asp, D). In particular, the invention relates to a cell comprising a DNA sequence which codes for the amino acid sequence shown in SEQ ID NO 12. One embodiment is an expression plasmid pLamanExp1 having the sequence shown in SEQ ID NO. 2.

One aspect of the invention relates to a method for the preparation of recombinant human LAMAN, the method comprising a) introducing, into a suitable vector, a nucleic acid fragment comprising a DNA fragment which codes for the amino acid sequence shown SEQ ID NO 12; b) transforming a cell with the vector obtained in step a); c) culturing the transformed host cell under conditions facilitating expression of the nucleic acid sequence; d) recovering the expression product from the culture.

The method may further comprise a fermentation step and/or a purification step. The invention further relates to a rhLAMAN produced by the method of the invention and to a method of treating a person having alfa mannosidosis, the method comprising administering a rhLAMAN produced by the method of the invention.

This application is a continuation-in-part of PCT/DK02/00388, filed Jun. 7, 2002 to be published in English, which is a non-provisional of 60/296,142, filed Jun. 7, 2001. All of the above-mentioned applications are hereby incorporated by reference in their entirety.

The present invention relates to a cell capable of producing recombinant human LAMAN (rhLAMAN), said cell comprising the 3066 basepair EcoRI-XbaI fragment of a human cDNA which codes for a human LAMAN protein in which the position corresponding to position 186 of the full length hLAMAN protein is Aspartic acid (Asp, D). In particular, the method relates to a cell comprising a DNA sequence which codes for the amino acid sequence shown in SEQ ID NO 12.

In a further aspect, the invention relates to a method for the preparation of recombinant human LAMAN, the method comprising a) introducing, into a suitable vector, a nucleic acid fragment comprising a DNA fragment which codes for the amino acid sequence shown SEQ ID NO 12; b) transforming a cell with the vector obtained in step a); c) culturing the transformed host cell under conditions facilitating expression of the nucleic acid sequence; d) recovering the expression product from the culture. The method may further comprise a fermentation step and/or a purification step.

Another embodiment of the present invention relates to a method for preventing or treating the development of symptoms related to alpha-mannosidosis caused by a deficiency, in a patient, of the human lysosomal alpha-mannosidase (hLAMAN) enzyme, the method comprising administering over a cellular membrane, to a target cell, an effective amount of recombinant human Lysosomal alpha-mannosidase (rhLAMAN) enzyme.

### BACKGROUND OF THE INVENTION

Alpha-Mannosidosis

Alpha-mannosidosis is a recessive autosomal disease that occur world wide with a frequency of between 1/1.000.000 and 1/500.000. Mannosidosis is found in all ethnic groups in Europe, America, Africa and also Asia. It is detected in all countries with a good diagnostic service for lysosomal storage disorders, at a similar frequency. They are born apparently healthy, however the symptoms of the diseases are progressive. Alpha-mannosidosis displays clinical heterogeneity, ranging from very serious to very mild forms. Typical clinical symptoms are: mental retardation, skeletal changes, impaired immune system resulting in recurrent infections, hearing impairment and often the disease is associated with a typical facial characteristics such as a coarse face, a prominent forehead, a flattened nasal bridge, a small nose, and a broad mouth. In the most severe cases (mannosidosis type I) the children suffer from hepatosplenomegaly, and they die during the first years of life. Possibly this early death is caused by severe infections due to the immunodeficiency caused by the disease. In milder cases (mannosidosis type 2) the patients usually reach adult age. The skeletal weaknesses of the result in the needs of wheeling chairs at age 20 to 40. The disease causes a diffuse dysfunction of the brain often resulting in weak mental performances that excludes anything but the most basic skills of simple reading and writing. These problems associated with hearing inabilities and other clinical manifestations preclude the patient from an independent life, the consequence being that life long caretaking is needed.

Lysosomal Alpha-Mannosidase

Alpha-mannosidosis results from a deficient activity of lysosomal alpha-mannosidase (LAMAN, EC3.2.1.24). The disease is characterised by massive intracellular accumulation of mannose-rich oligosaccharides. LAMAN is an exoglycosidase which hydrolyses terminal, non-reducing alpha-D-mannose residues in alpha-D-mannosides from the non-reducing end during the ordered degradation of the N-linked glycoproteins (Aronson and Kuranda FASEB J 3:2615-2622. 1989). The human enzyme is synthesised as a single polypeptide of 1011 amino acids with a putative signal peptide of 49 residues that is processed into three main glycopeptides of 15, 42, and 70 kD. (Nilssen et al. Hum.Mol.Genet. 6, 717-726. 1997).

The Lysosomal Alpha-Mannosidase Gene

The gene coding for LAMAN (MANB) is located at chromosome 19 (19cen-q12), (Kaneda et al. Chromosoma 95:8-12. 1987). MANB consists of 24 exons, spanning 21.5 kb (GenBank accession numbers U60885-U60899; Riise et al. Genomics 42:200-207. 1997). The LAMAN transcript is >>3,500 nucleotides (nts) and contains an open reading frame encoding 1,011 amino acids (GenBank U60266.1).

The cloning and sequencing of the human cDNA encoding LAMAN has been published in three papers (Nilssen et al. Hum.Mol.Genet. 6, 717-726. 1997; Liao et al. J.Biol.Chem. 271, 28348-28358. 1996; Nebes et al. Biochem.Biophys.Res.Commun. 200, 239-245. 1994). Curiously, the three sequences are not identical. When compared to the sequence of Nilssen et al (accession #U60266.1) a TA to AT change at positions 1670 and 1671 resulting in a valine to asparitic acid substitution was found by Liao et al. and Nebes et al.

Mutations

In 1997 Nilssen et al. (Hum Mol Genet 6:717-726) reported the first disease causing mutation in the fibroblasts from two siblings with mannosidosis in a highly inbred Palestinian family. In these two patients, the cause of the disease is a point mutation at the gene, with the nucleotide Adenine substituted with Thymidin. Since then, several genetic causes to the disease have been found in man. Today mutations causing alpha-mannosidosis have been characterised in more than 60 patients, manly in Europeans. These mutations vary both in type, encompassing nucleotide substitutions, deletions and additions and spanning most of the 24 exons of the gene (Berg et al. Am J Genet 64: 77-88. 1999).

The consequences of the mutations on the protein level also vary, from amino acid substitutions that effect both the active site and the folding, to early frame shifts and premature stop codons, resulting in a completely inactive product.

The amino acid sequence and enzyme structure in a number of different species. When comparing species, many of the human and the bovine mutations found are located to a highly “conserved” region of the mannosidase polypeptide for several species like man, cattle, cat, whale, bird, cod and slime mould, indicating that these regions are very important for the enzyme function.

Despite the many different types of mutations, it seems that all the mutations cause the similarly complete loss of enzyme activity in cells. Thus, apparently, the variations of clinical severity's cannot be explained by variations in the enzyme activity. Rather, these variations are caused by other factors, being attributed to the general ability of the organism to buffer the obnoxious effects of the accumulating oligomannosides, and by the environments to prevent and to treat infections and other clinical symptoms in the patients. Since no patients appear to possess enzyme activity, alpha-mannosidosis may be regarded as a mild disease compared to many other lysosomal storage disorders, but similar to the other glycoproteinoses (asparytlglucosaminoria, fucosidosis, Schindler's disease, beta-mannosidosis, Salla's disease).

Based on this one may assume that even a very small level of enzyme activity will be sufficient for a normal phenotype within this category of diseases.

Diagnosis

The diagnosis of alpha-mannosidosis is currently is based on clinical evaluation, detection of mannose-rich oligosaccharides in urine, and direct measurements of alpha-mannosidase activity in various cell types, such as leukocytes, fibroblasts, and amniocytes (Chester et al., In: Durand P, O'Brian J (eds) Genetic errors of glycoprotein metabolism. Edi-Ermes, Milan, pp 89-120. 1982; Thomas and Beaudet. In: Scriver C R, Beaudet A L, Sly W A, Valle D (eds) The metabolic and molecular bases of inherited disease. Vol 5. McGraw-Hill, N.Y., pp 2529-2562. 1995).

Because the symptoms initially often are mild and the biochemical diagnosis is difficult, the diagnosis is frequently made late in the course of the disease. It is obvious that patients and their families would benefit substantially from an early diagnosis.

Animal Models

Also animals may develop alpha-mannosidosis.

Alpha mannosidosis has been described in cattle (Hocking et al. Biochem J 128:69-78. 1972), cats (Walkley et al. Proc. Nat. Acad. Sci. 91: 2970-2974, 1994), and guinea pigs (Crawley et al. Pediatr Res 46: 501-509, 1999). A mouse model was recently generated by targeted disruption of the alpha-mannosidase gene (Stinchi et al. Hum Mol Genet 8: 1366-72, 1999).

Like in humans alpha mannosidase seems to be caused by specific mutations in the gene coding for lysosomal alpha-mannosidase. Berg et al. (Biochem J. 328:863-870. 1997) reported the purification of feline liver lysosomal alpha-mannosidase and determination of its cDNA sequence. The active enzyme consists of 3 polypeptides, with molecular masses of 72, 41, and 12 kD. Similary to the human enzyme it was demonstrated that the feline enzyme is synthesized as a single-chain precursor with a putative signal peptide of 50 amino acids followed by a polypeptide chain of 957 amino acids, which is cleaved into the 3 polypeptides of the mature enzyme. The deduced amino acid sequence was 81.1% and 83.2% identical with the human and bovine sequences, respectively. A 4-bp deletion was identified in an affected Persian cat; the deletion resulted in a frameshift from codon 583 and premature termination at codon 645. No enzyme activity could be detected in the liver of the cat. A domestic long-haired cat expressing a milder phenotype had enzyme activity of 2% of normal; this cat did not possess the 4-bp deletion. Tollersrud et al. (Eur J Biochem 246:410-419. 1997) purified the bovine kidney enzyme to homogeneity and cloned the gene. The gene was organized in 24 exons that spanned 16 kb. Based on the gene sequence they identified two mutations in cattle.

Medical Need for Alpha-Mannosidosis Therapy

The lack of effective treatment for alpha-mannosidosis is well recognised.

Enzyme Substitution

When lysosomal storage diseases were discovered, hopes were raised that this could be treated by enzyme substitution. Enzyme replacement therapy has proven efficient in Gaucher disease. When exogenous lysosomal glucocerebrosidase is injected into the patient, this enzyme is taken up by enzyme-deficient cells (Barton et al. N Engl J Med 324:1464-1470). Such uptake is regulated by certain receptors on the cell surface. Different tissue have different receptors. It is therefore necessary to change the natural enzyme to enable uptake into the tissue of interest. If properly designed, the missing enzyme could be injected regularly like the diabetic subject injects insulin. In many in vitro studies with the purified active enzyme added to the media of enzyme-deficient fibroblasts, the lysosomal substrate accumulation was corrected, but in vivo treatment has been hampered through the problem of producing the sufficient quantity of enzymes, immunological problems, and that the substituted enzyme does not pass the blood-brain barrier.

Bone Marrow Transplantation

In 1996 Walkley et al. (Proc. Nat. Acad. Sci. 91: 2970-2974, 1994) published a paper on 3 kittens with mannosidosis that were treated with bone marrow transplantation (BMT) in 1991. In the 2 animals that were sacrificed a normalisation was seen, not only in the body, but more importantly, also in brain. The 3'rd cat was well after 6 years. Normally, an untreated cat dies within 3-6 months. In 1987 a child with mannosidosis was treated with BMT (Will et al. Arch Dis Child October 1987; 62(10):1044-9). He died after 18 weeks due to procedure related complications. In brain little enzyme activity was found. This disappointing result could be explained by heavy immunosuppressive treatment before death, or that it takes time for the enzyme activity to increase in brain after BMT. The donor was the mother (who as carrier must be expected to have less than 50% enzyme activity) or it may be BMT in man has no effect on enzyme function in brain.

### DISCLOSURE OF THE INVENTION

A particularly important embodiment of the present invention is a cell capable of producing recombinant human LAMAN (rhLAMAN), said cell comprising the approximately 3066 basepair EcoRI-XbaI fragment of a human cDNA which codes for a human LAMAN protein in which the position corresponding to position 186 of the full length hLAMAN protein is Aspartic acid (Asp, D). In a recent paper Berg et al. (Mol Gen and Metabol 73, 18-29, 2001) described a cell capable of producing rhLAMAN. Whereas Berg et al. does not disclose the sequence of the particular cDNA used in the paper the same group has published their LAMAN sequence as GenBank accession no. U60266. In this sequence the amino acid corresponding to position 186 is Valine not Aspartic acid as in the present application. Interesting, Nebes et al. (Biochem.Biophys.Res.Commun. 200, 239-245. 1994) reports that the amino acid at position 186 is Aspartic acid. However, the sequence of Nebes et al. differs from the sequence disclosed herein at many other positions. For instance the position corresponding to position 343 in the amino acid sequence is missing in Nebes et al., also the start of the protein sequence disclosed herein is absent in Nebes et al.

A further preferred embodiment of the present invention is a cell which contains a DNA fragment comprising the approximately 3066 basepair EcoRI-XbaI fragment of the DNA fragment having the sequence shown in SEQ ID NO 2. In addition to coding for full length hLAMAN the sequence is engineered to comprise two convenient restriction enzyme sites for easy insertion into a wide range of suitable expression vectors.

A most preferred embodiment of the present invention is a cell obtained by use of the expression plasmid pLamanExp1 since this plasmid contains signal sequences necessary to obtain high expression levels and such a cell furthermore is suitable for amplification as described in example 2.

Another inventive concept of the present invention is based on the novel idea of substituting the reduced hLAMAN enzymatic activity in the person having alpha-mannosidosis simply by administering over a cellular membrane, to a target cell, an effective amount of an hLAMAN enzyme. This can thereby “assist” the enzyme that is in deficit.

Accordingly, the present invention relates to a method for preventing or treating the development of symptoms related to alpha-mannosidosis(alpha-mannosidosis) caused by a deficiency, in a subject, of the human Lysosomal alpha-mannosidase (hLAMAN) enzyme, the method comprising administering over a cellular membrane, to a target cell, an effective amount of an hLAMAN catalyst which is said enzyme or an enzymatically equivalent part or analogue thereof.

Since mannose-rich glycoproteins accumulates in all cells of the body and causes the symptoms of mannosidase, the target cells for hLAMAN therapy are all cells of the body. Since the some of the most severe symptoms caused by hLAMAN deficiency are related to the central nervous system (CNS) the CNS including the brain is the one important objective of the enzyme replacement therapy. However, beneficial delivery of hLAMAN to the brain requires that the hLAMAN is able to cross the blood-brain-barrier (BBB).

In a preferred embodiment, the present invention involves a treatment method in which a cellular barrier such as the blood-brain-barrier (BBB) is crossed whereby the material is delivered to the target cells.

Effective enzyme replacement therapy of alpha-mannosidosis patients with recombinant human LAMAN (rhLAMAN) will require the uptake of an active enzyme into the target cells, and furthermore that the rhLAMAN inside the cell is transported to the lysosomes. As described this can be accomplished by tagging the rhLAMAN. The enzyme may e.g. be tagged with mannose. E.g. endothelial cells have a mannose receptor mediated protein uptake, the result being that mannose tagged proteins are internalised by liver endothelial cells, but not by cells that do not have this receptor. A number of other tags are conceivable. One particular interesting type of tag is the mannose-6-phosphate (M-6-P). This tag is particular interesting since all cell-types contain this receptor, which is cirkulating between the Golgi apparatus, the endosomes and the plasma membrane. Extracellular enzymes are captured by the M-6-P receptor found on the cell surface and be internalized and delivered to the lysosomes (Willingham, et al., 1981, Proc. Natl. Acad. Sci. U.S.A. 78: 6967). It is established that the secreted forms of lysosomal enzymes produced in mammalian cell lines, for example CHO cells, contain the mannose-6-phosphate tag. This should also be valid for lysosomal alpha-mannosidase, since it is normally transported to the lysosomes by a mannose-6-phosphate dependent pathway.

However, enzyme replacement therapy is not effective against the acute neuronopathic variant of Gaucher disease (Prows et al. Am J Med Genet 71:16-21). To be able to deliver rhLAMAN to the brain a vehicle that can facilitate passage of the blood-brain-barrier (BBB) is needed since rhLAMAN is not likely to be able to traverse over the BBB by it self. Such “vehicle” can be a polyamine-modified, a peptide-modified, an antibody-modified LAMAN or even a cell.

### DEFINITIONS

By the term “catalyst” is herein meant either the relevant enzyme which is substituted as it is, or an enzymatically equivalent part or analogue thereof. One example of an enzymatically equivalent part of the enzyme could be a domain or sub-sequence of the enzyme which includes the necessary catalytic site to enable the domain or sub-sequence to exert substantially the same enzymatic activity as the full-length enzyme. An example of an enzymatically equivalent analogue of the enzyme could be a fusion protein which includes the catalytic site of the enzyme in a functional form, but it can also be a homologous variant of the enzyme derived from another species. Also, completely synthetic molecules, which mimic the specific enzymatic activity of the relevant enzyme, would also constitute “enzymatic equivalent analogues”.

By the term “hybrid molecule” is herein meant a fusion protein between the mhLAMAN catalyst and the peptide required for transport of the vehicle over the BBB and/or cellular membranes. In the said fusion protein the vehicle part can be attached to either the C-terminal or N-terminal end of the mhLAMAN catalyst.

By the term “recombinant techniques” is herein meant a technique which involves recombinant DNA molecules, that is hybrid DNA sequences comprising at least two DNA sequences, the first sequence not normally being found together in nature with the second.

By the terms “recombinant polynucleotide” or “recombinant polypeptide” as used herein are meant at least a polynucleotide or polypeptide which by virtue of its origin or manipulation is not associated with all or a portion of the polynucleotide or polypeptide with which it is associated in nature and/or is linked to a polynucleotide or polypeptide other than that to which it is linked in nature.

The term “cloning vehicle” describes a plasmid, virus, retrovirus, bacteriophage, cosmid, artificial chromosome (bacterial or yeast), or nucleic acid sequence which is able to replicate in a host cell, characterized by one or a small number of restriction endonuclease recognition sites at which the sequence may be cut in a predetermined fashion, and which may contain an optional marker suitable for use in the identification of transformed cells, e.g., tetracycline resistance or ampicillin resistance. A special class of cloning vectors possess the features necessary for it to operate as an expression vector. An “expression vector” has a promoter positioned upstream of the site at which the sequence is cut for the insertion of the heterologous DNA sequence, the recognition site being selected so that the promoter will be operatively associated with the heterologous DNA sequence. A heterologous DNA sequence is “operatively associated” with the promoter in a cell when RNA polymerase which binds the promoter sequence transcribes the coding sequence into mRNA which is then in turn translated into the protein encoded by the coding sequence.

By the term “target cell” is herein meant a cell or group of cells (tissue) to which the enzymes should be delivered.

### DETAILED DISCLOSURE OF THE INVENTION

The present invention relates to a cell capable of producing recombinant human LAMAN (rhLAMAN), said cell comprising the 3066 basepair EcoRI-XbaI fragment of a human cDNA which codes for a human LAMAN protein in which the position corresponding to position 186 of the full length hLAMAN protein is Aspartic acid (Asp, D). In particular, the invention relates to a cell comprising a DNA sequence which codes for the amino acid sequence shown in SEQ ID NO 12.

A specific embodiment of the invention relates to a cell obtained by insertion of a DNA fragment comprising the 3066 basepair EcoRI-XbaI fragment of the DNA fragment having the sequence shown in SEQ ID NO 2, in particular a cell obtained by use of the expression plasmid pLamanExp1 having SEQ ID NO. 2. The cell according to the invention may be obtained by transfection of a non-human mammalian cell line such as by transfection of chinese hamster ovary (CHO) cells.

A presently preferred embodiment of the invention relates to a cell obtainable by the culture of the human LAMAN production cell line DSM ACC2549 which has been deposited at the DSMZ for the purposes of patent deposit according to the Budapest Treaty on Jun. 6, 2002. The invention further relates to an expression plasmid pLamanExp1 having the sequence shown in SEQ ID NO. 2.

In another embodiment, the invention relates to a method for the preparation of recombinant human LAMAN, the method comprising a) introducing, into a suitable vector, a nucleic acid fragment comprising a DNA fragment which codes for the amino acid sequence shown SEQ ID NO 12; b) transforming a cell with the vector obtained in step a); c) culturing the transformed host cell under conditions facilitating expression of the nucleic acid sequence; d) recovering the expression product from the culture.

The method may further comprise a fermentation step and/or one or further purification steps.

In particular, the invention relates to a method wherein the nucleic acid fragment comprises the 3066 basepair EcoRI-XbaI fragment of the DNA fragment shown in SEQ ID NO 2.

An important aspect of the invention relates to a rhLAMAN produced by the method of the invention and to the use of the rhLAMAN according to the invention for the preparation of a medicament for the treatment of alfa mannosidosis.

In its broadest aspect, the present invention relates to a recombinant polypeptide and to a nucleotide sequence encoding the LAMAN polypeptide, to an expression system capable of expressing the polypeptide as well as to pharmaceutical compositions comprising the polypeptide or part of it and to the use of the polypeptide for preventing or treating the development of symptoms related to alpha-mannosidosis(alpha-mannosidosis) caused by a deficiency, in a subject, of the human Lysosomal alpha-mannosidase (hLAMAN) enzyme.

Preferably, the hLAMAN catalyst is administered over a cellular membrane, to a target cell, by uptake of hLAMAN into the target cell by taking advantage of a mannose- or M-P-6-receptor-mediated uptake.

Mannose-6-phosphate tagged hLAMAN is preferably made in a mammalian cell system (e.g. CHO, COS cells or BHK cells (Stein et al. J Biol Chem.1989, 264, 1252-1259) to secure correct mannose-6-phosphate tagging on the molecule, which ensures efficient receptor mediated uptake. Mannose-6-phosphate tagged mhLAMAN is secreted into the medium and purification of rhhLAMAN may be facilitated by the use of ammonium salts (NH₄Cl) in the fermentation step.

Example 3 provides an example of such production of mannose-6-phosphate tagged hLAMAN

The production of recombinant hLAMAN which is mannose-6-P comprises one or several or all of the following steps (an outline):

A. Synthesis of rhLAMAN

Cloning of specific rhLAMAN

B. Transfection

2-10 μg rhLAMAN hybrid vector DNA is used for transfection by phosphate precipitate/glycerol shock methodology, into mammalian cells (e.g. CHO, COS cells or BHK cells). Transfection might also be done with an electric shock methodology.

C. Expression of rhLAMAN

Synthesis of the active protein and the mannose-6-P modification of said protein is done during the expression in the mammalian cell system.

D. Purification of rhLAMAN

rhLAMAN is purified using a 6 step procedure—see example 3.

E. Test System for Mannose-6-P Receptor Mediated Uptake

The ability of produced rhLAMAN to be active in a mannose-6-P receptor mediated uptake is tested by incubating rhLAMAN with normal fibroblasts or fibroblasts from alpha-mannosidosis patients. Uptake into cells is assayed by increased hLAMAN activity.

Example 3 shows an example of mannose-receptor-mediated uptake of hLAMAN into cells.

In the following different delivery techniques of LAMAN enzyme across the BBB and/or cellular membranes are described.

1) Peptides and proteins as vehicles for passage LAMAN to the target cells by passage over cell membranes and/or the BBB:

A number of studies in animals have shown that certain proteins and/or peptides may act as vehicles for passage of BBB. For instance proteins modified by the insulin fragment (Fukuta et al. Phaomacol Res 11: 1681-1688) or antibodies to the transferrin receptor (Friden et al. Proc Natl Acad Sci USA 88: 4771-4775) can pass the blood-brain barrier. Also proteins modified by coupling to polyamines (Poduslo and Curran. J Neurochem 66: 1599-1606) have been reported to pass the blood-brain barrier.

2) Toxins as vehicles for passage LAMAN to the target cells by passage over cell membranes and/or the BBB:

Different bacteria, plants and animals produce toxins. Toxins have many different targets such as the gut (enterotoxins), nerves or synapses (neurotoxins). Toxins can traverse cell membranes via receptor mediated processes and the embodiment of the present invention is to use toxins as vehicles to passage rhLAMAN to the target cells over cellular membranes and/or the BBB. The preferred target cells are cells in the CNS and/or the peripheral nervous system.

A further embodiment of the present invention is that only the peptide pertaining to the translocation over cellular membranes and/or the BBB of the toxin is used.

One example of a toxin used as a vehicle is a bacterial toxin such as Diphtheria Toxin (DT), from the Corynebacterium Diptheriae. Bacterial toxins exhibit a wide range of toxicities and they fall into groups by structure and function. The toxin binds to a target cell and enters the cell via a receptor, and is reduced to separate fragments. The processed toxin can be divided into the following 3 domains: The catalytic domain (C), the receptor domain (R), and the translocation domain (T).

The catalytic fragment and the receptor fragment of the toxin or fragments thereof are replaced by the LAMAN. This fusion protein can traverse cellular membranes and/or the BBB and thereby deliver the LAMAN to the target cells. One example of the engineering of a hybrid molecule could be by recombinant technology.

A. Synthesis of rhLAMAN

Cloning of specific rhLAMAN cDNA from human liver, placenta or spleen library.

B. Synthesis of DT cDNA

1. Cloning of Diphtheria Toxin cDNA.

2. Removal of the cDNA coding for the catalytic domain and the receptor domain or fragments thereof. The remaining fragment of DT is in the following termed “DT(T).

C. Construction of the DT(T)-rhLAMAN Hybrid

1. Ligation of rhLAMAN cDNA with the DT(T) fragment.

D. Expression

1. Ligate the DT(T)-rhLAMAN construct in a vector for efficient expression in a mammalian cell system e.g. COS, BHK or CHO.

2. Conversion of the rLAMAN to mannose-6-P tagged protein occur during the expression in the mammalian cell system.

Other examples of bacterial toxins to be used as vehicles are Clostridium Botulinum, Pseudomonas Exotoxin A produced by Psedomonas aeruginosa, Cholera Toxin produced by Vibrio cholerae, and Pertussis Toxin produced by Bordetella pertussis.

Further examples of toxins used as vehicles are plant toxins selected from the list of the following plant toxins: cholinesterase inhibitors, protease inhibitors, amylase inhibitors, tannins, cyanogenic glycosides, goitrogens, lectin proteins, and lathyrogens, pyrrozidine alkaloids.

Yet further examples of toxins used as vehicle are toxins from shellfish (saxitoxin) and snakes (alpha-bungarotoxin). The skilled person may add further examples in light of the details and characteristics of the invention.

3) Proteins and/or peptides isolated from bacteria or viruses as vehicles for passage rhLAMAN to the target cells by passage over cell membranes and/or the BBB:

The transacting element and/or transacting protein from bacteria or viruses can be used together with rhLAMAN to cross the BBB and/or cellular membranes.

Examples of bacteria are selected from the following list: Ns. meningitidis, S. pneumoniae, Hemophilus influenzae, Staphylococcus species, Proteus species, Pseudomonas species, E. coli, Listeria monocytogenes, M. Tuberculosis, Neurolues, Spirochetes Borrelia burgdorferi from Iodex ricinus.

Examples of viruses are selected from the following list of virus families: Parvoviridae, Papovaviridae, Adenoviridae, Herpesviridae, Poxviridae, Picornaviridae, Reoviridae, Togaviridae, Arenaviridae, Coronaviridae, Retroviridae, Bunyaviridae, Orthomyxoviridae, Paramyxoviridae, and Rhabdoviridae. Relevante examples of viruses selected from the list mentioned above are e.g. Measles virus, Papova virus, and JC virus.

4) Mannose-receptor mediated uptake of hLAMAN into cells:

Several human cells (monocytes, fibroblasts, lymphocytes) have shown to be able to cross the BBB by it-self (Hickey W F, Kimura H. Science. 1988, 239, 290-292, Hickey W F et al. J Neurosci Res. 1991, 28, 254-260). These cells can be “loaded” with rhLAMAN and can act as a vehicle for transport of rhLAMAN to the brain. Preferably, lymphocytes are used as vehicles, because they have a long half-life (2-3 months). Uptake of rhLAMAN into lymphocytes will take advantage of a mannose-receptor-mediated uptake.

Mannose-6-phosphate tagged rhLAMAN is made in a mammalian cell system (e.g. CHO, COS cells or BHK cells (Stein et al. J Biol Chem.1989, 264, 1252-1259) to secure correct mannose-6-phosphate tagging on the molecule, which ensures efficient receptor mediated uptake. Mannose-6-phosphate tagged rhLAMAN is secreted into the medium and purification of rhLAMAN may be facilitated by the use of ammonium salts (NH₄Cl) in the fermentation step.

The disease which is the target for the inventive method is alpha-mannosidosis, and therefore the catalyst is hLAMAN or an enzymatically equivalent part or analogue thereof. It is most preferred that the catalyst is a recombinant form of the human hLAMAN enzyme or of the enzymatically equivalent part or analogue thereof, since recombinant production will allow large-scale production which, with the present means available, does not seem feasible if the enzyme would have to be purified from a native source.

Furthermore, use of such catalysts, as described herein, for the preparation of pharmaceutical compositions for treatment of the above-discussed diseases is also part of the invention.

Thus, the present invention represents an important advance in the treatment of genetic and/or acquired metabolic brain disorders of alpha-mannosidosis problems associated with prior treatment methods, including the gene therapy and bone marrow transplantation.

The present invention also relates to the new and surprising concept of use as a carrier of a peptide or protein from a structure capable of crossing a biological barrier, such as a cellular barrier including the blood-brain-barrier or a specific membrane of a cell. The object of the carrier function is to deliver an enzyme to a target cell. The target cell is generally a cell wherein the enzyme activity is insufficient either due to an decrease activity of the enzyme or to a situation where an increased activity is desired.

In one embodiment, the invention relates to a method for increasing the content of an enzyme in a cell comprising delivery of the enzyme to the tissue relevant for the cell and/or to the cell by use of a protein or peptide capable of crossing a cellular barrier, the protein or peptide being derivable from the group of toxins, bacteria, from viral peptides, and from fragments and modifications thereof.

The disease alpha-mannosidosis has been explained in detail above and the present invention is of particular importance in connection with alleviating the progression of symptoms caused by the enzyme defect of hLAMAN. Accordingly, in a preferred embodiment, the invention relates to a method for preventing or treating the development of symptoms related to alpha-mannosidosis comprising the administration of the enzyme recombinant human lysosomal alpha-mannosidase (rhLAMAN) to the tissue relevant for the cells and/or to the cells by use of a protein peptide capable of crossing a cellular barrier or by use of a human cell as a vehicle for delivering of the enzyme to the tissue relevant for the cells and/or to the cells.

Similar to the use of a proteins such as insulin or transferrin antibodies as carrier proteins, the use of a protein or peptide derivable from a toxin protein has been suggested as a carrier protein the present invention. Such toxins may be a toxin selected from plant toxins, bacterial toxins and from toxins from animals. The toxin may be modified in order to increase the desired properties of the peptide.

In one aspect, a protein or peptide derivable from a virus may be used. However, as virus in its nature has the cell nucleus as target organ for the infection, it is contemplated that a modification or specific fragment not having this effect may be a preferred embodiment.

However, the delivery of an enzyme to an enzyme deficient cell related to alpha-mannosidosis by use of an protein virus is a further surprising aspect of the invention.

As virus in general is an inhomogeneous group with respect to affinity for different cells, it is within the spirit of the present invention to select the concrete virus in accordance with the desired target cell to which the enzyme is to be delivered.

Affection of the nervous system, especially of the central nervous system, very often plays a significant role in the broad range of diseases caused by enzyme deficiency. Accordingly, in a preferred embodiment, the carrier protein or peptide is one which is capable of crossing the blood-brain-barrier.

This is possible by use of proteins or peptide which due to their nature bears a structure relevant for such transduction. In an important method according to the invention, the cellular barrier includes the blood-brain-barrier (BBB) and the tissue is a tissue and/or cell of the central nervous system.

The overall idea of the present invention is to prepare a construct comprising the enzyme in question the delivering protein/peptide. Accordingly, in one aspect the invention relates to a method wherein the enzyme forms a hybrid with the protein or peptide capable of crossing the cellular barrier.

As explained in further details in the text of the present specification the hybrid is preferably produced by recombinant techniques. However, the construct may be produced by techniques of protein synthesis generally known in the art including solid phase synthesis. The complete hybrid or part of the hybrid may accordingly be produced synthetically or a part or the hybrid construct may be produced by use of a genuine protein or peptide. The enzyme part and delivery part may be linked by different techniques known in the art.

Preferably, the hLAMAN is made by recombinant techniques. In a further embodiment, the LAMAN is human (hLAMAN) and still more preferred mature human hLAMAN (mhLAMAN) or a fragment thereof. The fragment may be modified, however the active sites of the enzyme should be preserved.

In a still further aspect of the invention, cells are used as a vehicle for delivering an enzyme, preferably hLAMAN to the target cell. The preferred human cell is selected from human monocytes, human fibroblasts, and human lymphocytes. As explained above, it is preferred for target cells of the central nervous system that the cell for delivering the enzyme is capable of crossing the BBB for delivering the hLAMAN to the tissue and/or cells of the central nervous system.

In order to facilitate the delivery of the enzyme to the target cell, the hLAMAN may be transferred to a target cell by means of a mannose-receptor-mediated uptake. Such uptake may be further increased when the hLAMAN is a mannose-6-P tagged hLAMAN, preferable made by expressing hLAMAN in a mammalian cell system. Preferred mammalian cell system is selected from CHO cells, COS cells, and BHK cells.

In general, the target cell is a cell wherein the activity, such as hLAMAN is insufficient for the optimal function of the cell. Insufficient activity of hLAMAN may be measured by one or more of the parameters selected from monitoring incresed levels of mannose-rich oligosaccharides in urine, analysis of hLAMAN activity in material from the patient such as in leukocytes and/or in skin fibroblasts, presence of clinical symptoms or increase in rate of development of clinical symptoms of alpha-mannosidosis.

A significant feature of insufficient hLAMAN activity is a cell wherein a massive intracellular accumulation of mannose-rich oligosaccharides is present. Naturally, such cell is a target cell according to the present invention. The target cell may also be cells of the nervous system. In addition, target cells for delivering the enzyme also includes one or more cell types selected from human monocytes, human fibroblasts, human lymphocytes, human macrophages.

An increased activity of the hLAMAN may be used as a parameter for a treatment schedule an may be measured by one or more of the parameters selected from monitoring incresed levels of mannose-rich oligosaccharides in urine, analysis of hLAMAN activity in material from the patient such as in leukocytes and/or in skin fibroblasts, presence of clinical symptoms or increase in rate of development of clinical symptoms of alpha-mannosidosis.

It is a very important aspect of the invention to perform the treatment of a possible enzyme defect prenatally. In a further aspect of the invention, the cellular membrane is the fetal-maternal barrier (placenta). It is also within the scope of the invention to deliver the enzyme-protein-construct directly to the fetus prenatally.

In a still further embodiment, the invention relates to an antibody raised against any of the constructs formed by any of the enzymes and any of the proteins and/or peptide mentioned herein. Such antibody may be used for the targeting of the construct, for inactivation of the construction including increasing the elimination of the construct from the subject or even more important for detecting or monitoring the level of intracellular levels of LAMAN. The antibody may be a polyclonal antibody or a monoclonal antibody and by be produced by techniques known in the art.

Any enzyme construct disclosed above as well as the antibody thereto may be use for the preparation of a medicament for a treatment in accordance with the method disclosed in the present context. Accordingly, the present invention also relates to a pharmaceutical medicament comprising an enzyme linked to a carrier system such as a protein or peptide or to a cell system as disclosed in detail above.

Accordingly, in one aspect, the present invention relates to a construct as well as to the use of a construct comprising an enzyme and/or a protein or peptide protein peptide capable of crossing a cellular barrier or a human cell as a vehicle for delivering of the enzyme to the tissue relevant for the cells and/or to the cells as disclosed in any of the methods mentioned and specified in the claims for the preparation of a medicament.

Also the antibody and use thereof the preparation of a medicament is within the scope of the invention.

In a presently preferred aspect, the present invention relates to the use of the LAMAN coding sequence of FIG. 4 for the use in gene therapy. It is suggested that a recombinant viral or non-viral vector comprising the cDNA coding for the biologically active human LAMAN and able to infect and/or transfect and sustain expression of the biologically active human LAMAN in mammalian cells could be produced. Such constructs could be used to treat alpha-mannosidase patients.

In a very one important aspect the present invention the principle of “gene therapy” is developed in relation to therapy by bone marrow transplantation. Bone marrow transplantation (BMT) with cells that do produce LAMAN is known to have a curative effect on individuals (test animals) suffering from alpha-mannosidosis (Walkley et al. Proc. Nat. Acad. Sci. 91: 2970-2974, 1994). BMT may even be beneficial to human patients. In 1987 a child with mannosidosis was treated with BMT (Will et al. Arch Dis Child October 1987; 62(10):1044-9). He died after 18 weeks due to procedure related complications. This disappointing result could be explained by heavy immunosuppressive treatment before death. This treatment was necessary because the patient received non-self bone-marrow cells. One way to overcome the problem of rejection of foreign bone marrow cells could be to use the patients own cells. We suggest to transfect such stem cells with a construct comprising the LAMAN cDNA nucleic acid sequence to obtain bone marrow stem cells that express enhanched levels of LAMAN, and finally to reintroduce these cells into the bone marrow of the patient.

### LEGEND TO FIGURES

FIG. 1.

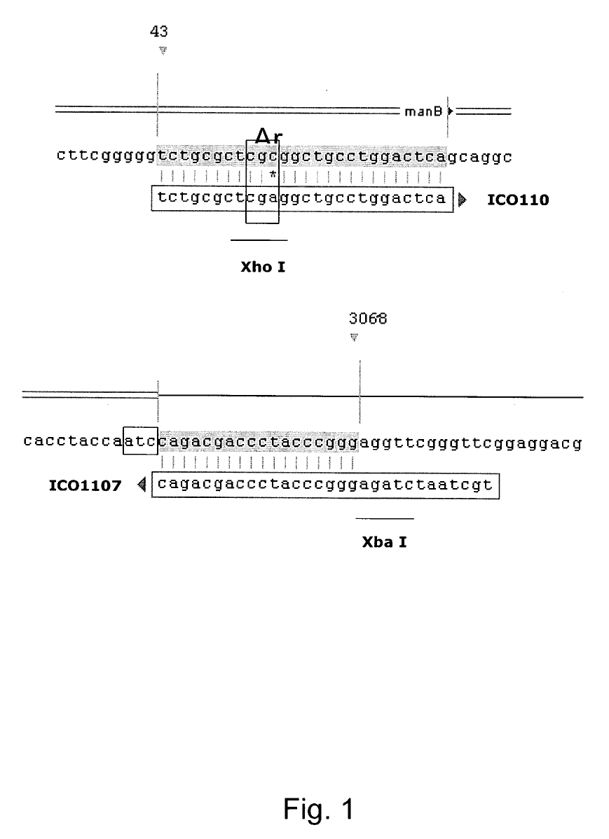

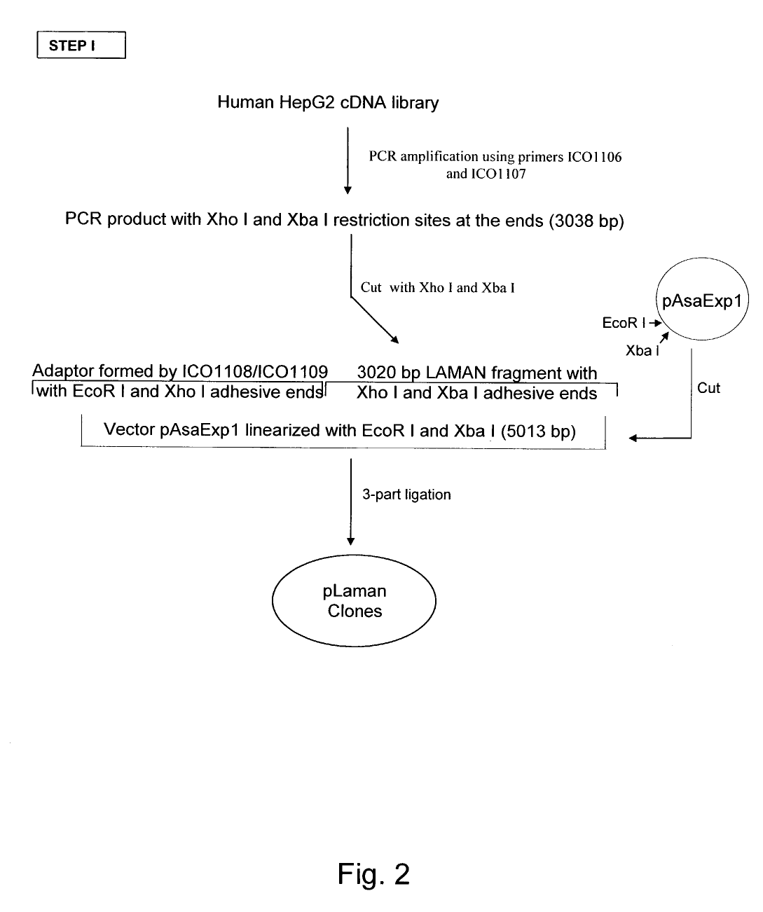

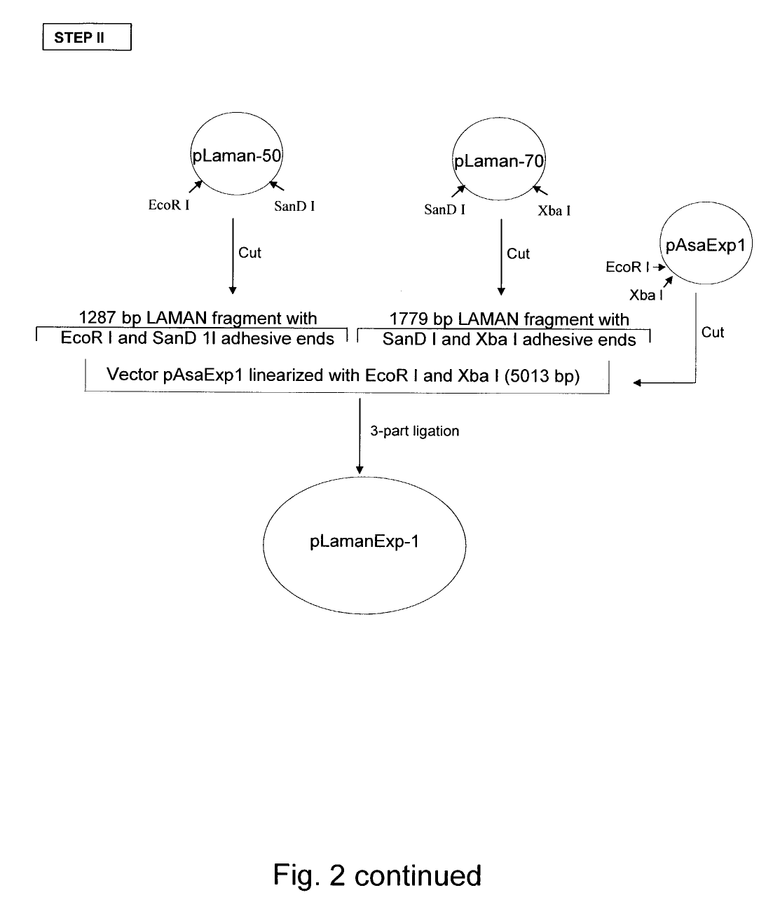

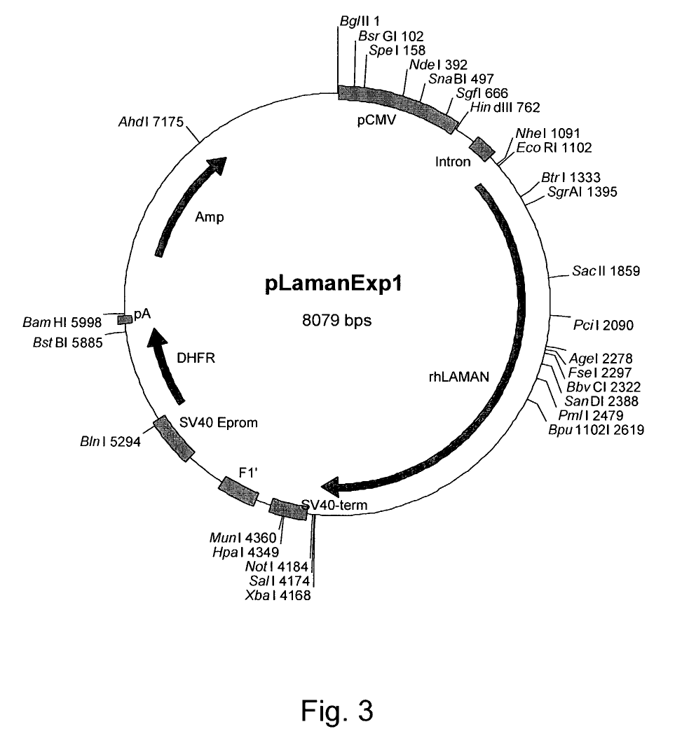

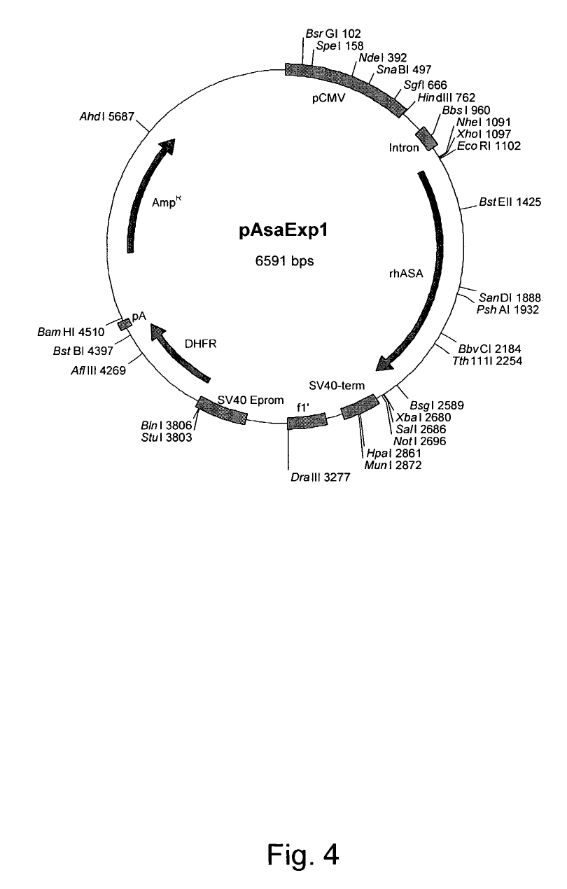

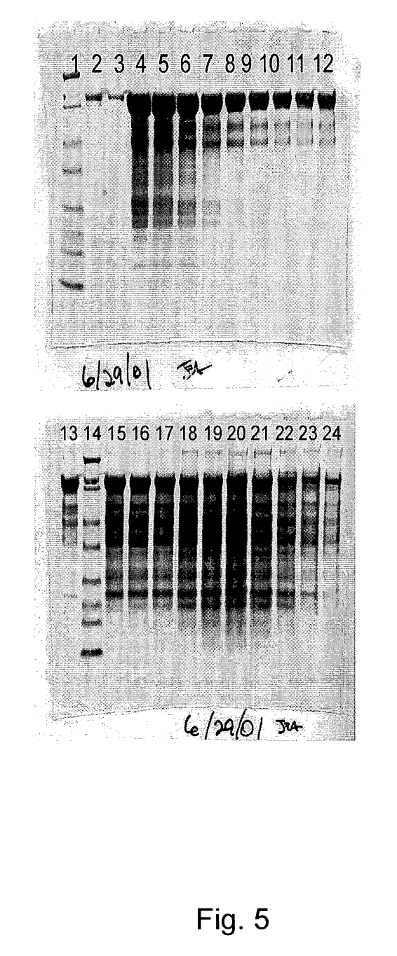

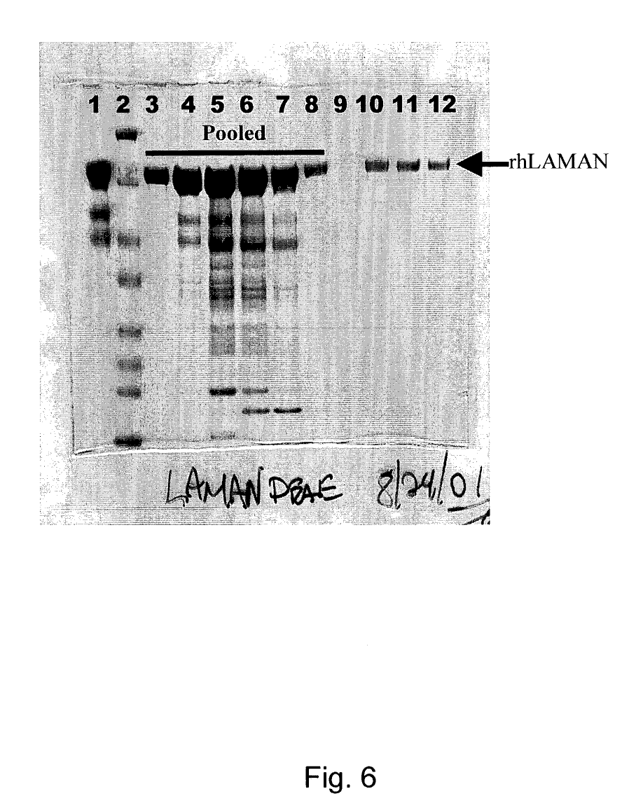

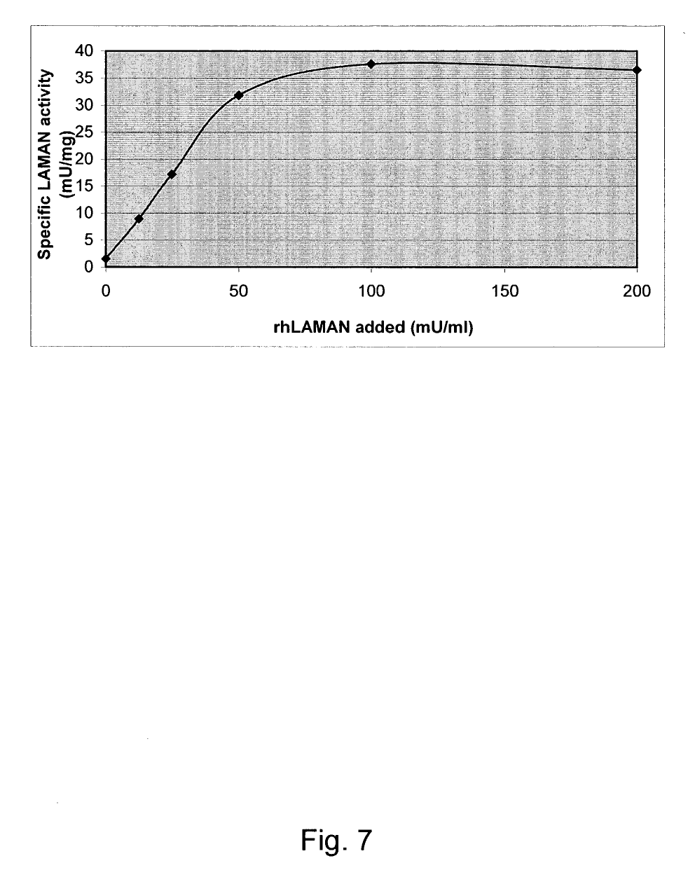

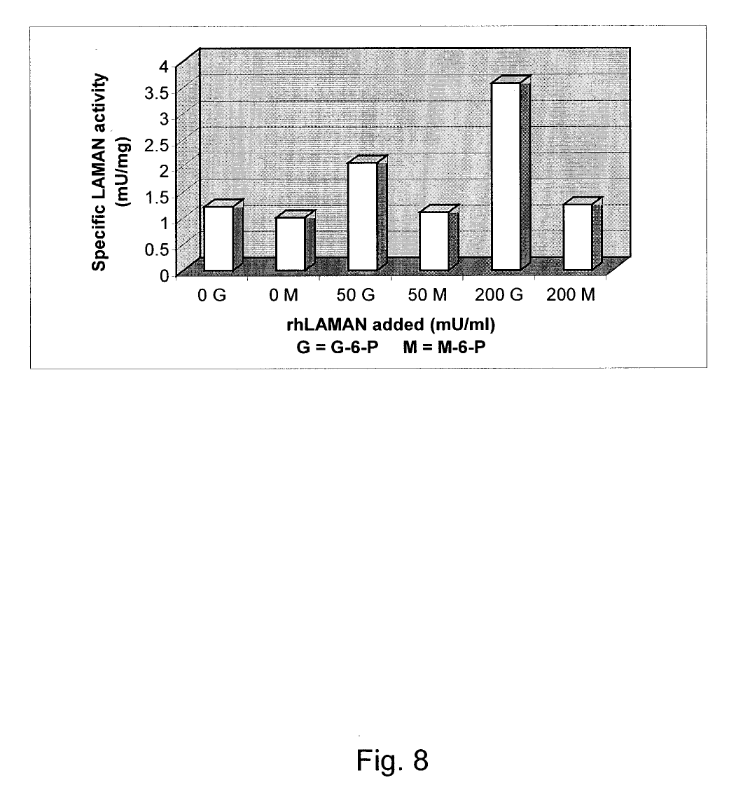

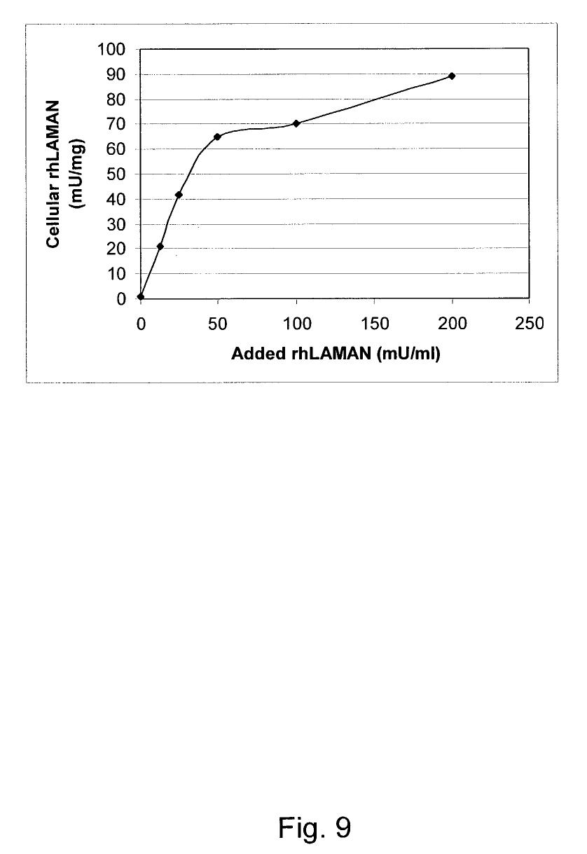

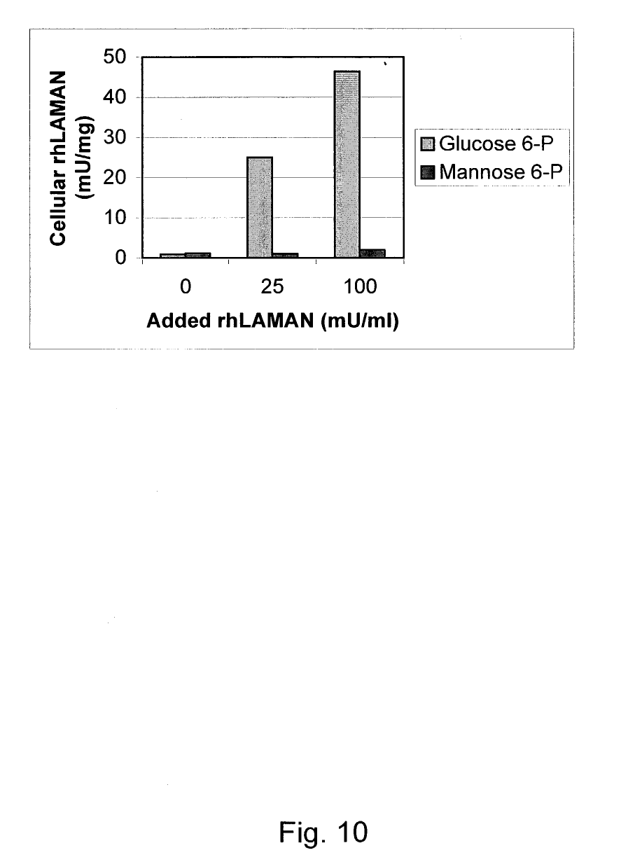

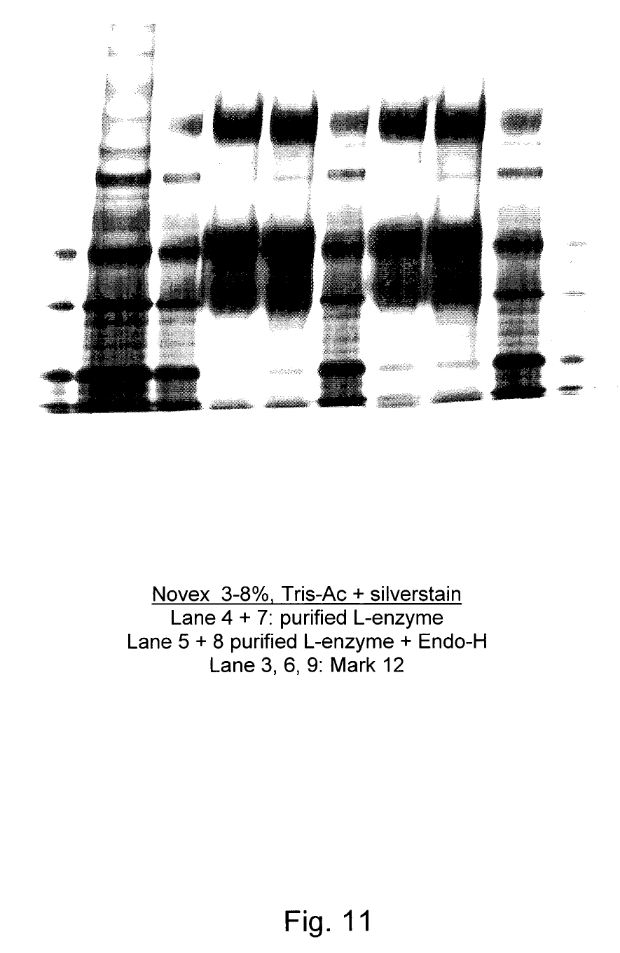

Description of oligonucleotides used for PCR amplification.

The two oligonucleotides ICO1106 and ICO1107 are shown below the LAMAN sequence. Numbering refers to the numbering in the GenBank sequence Accession #U60266.1. The highlighted regions in the LAMAN sequence represent the regions of homology with the oligonucleotide primers. The asterisk denotes the C to A change introduced at position 53 to generate a Xho I site for ease of manipulation. This is a silent mutation in the codon for the arginine residue at position 13 in the amino acid sequence of the α-mannosidase protein. The triplets coding for this arginine residue and for the termination codon are boxed. The PCR primer ICO1107 has a 5′ extension incorporating a Xba I site to allow cloning of the amplified DNA.

FIG. 2.

Strategy for PCR cloning of LAMAN.

FIG. 3.

Map of the expression plasmid pLamanExp1.

“pCMV” is the Human cytomegalovirus immediate-early promoter/enhancer. “Intron” is a chimeric Intron. “rhLAMAN” is the recombinant human lysosomal alpha monnosidase coding region. “SV40-term” is the SV40 late termination and polyadenylation signal. “F1′” is Phage f1 region. “SV40 Eprom” is the SV40 early promoter/enhancer and origin. “DHFR” is the Mouse dihidrofolate reductase coding region. “pA” is a synthetic polyadenylation signal. “AmpR” is the beta-lactamase (AmpR) coding region.

FIG. 4.

Map of the expression plasmid pAsaExp1.

“pCMV” is the Human cytomegalovirus immediate-early promoter/enhancer. “Intron” is a chimeric Intron. “rhASA” is the recombinant human Arylsulfatase A coding region coding region. “SV40-term” is the SV40 late termination and polyadenylation signal. “F1′” is Phage f1 region. “SV40 Eprom” is the SV40 early promoter/enhancer and origin. “DHFR” is the Mouse dihidrofolate reductase coding region. “pA” is a synthetic polyadenylation signal. “AmpR” is the beta-lactamase (AmpR) coding region.

FIG. 5. SDS-PAGE analysis of anion exchange chromatography for rhLAMAN. See table 2 or content of each lane.

FIG. 6. SDS-PAGE analysis of anion exchange chromatography for rhLAMAN (larger scale). See table 3 for content of each lane.

FIG. 7. Fibroblasts from a patient with mannosidosis (GM00654) loaded with DEAE-purified rhLAMAN for 40 hours in complete medium.

FIG. 8. Fibroblasts from a patient with mannosidosis (GM00654) loaded with DEAE-purified rhLAMAN for 40 hours in complete medium with the addition of 5 mM Mannose-6-Phosphate (M-6-P) or 5 mM Glucose-6-Phosphate (G-6-P).

FIGS. 9 and 10. Cellular uptake of rhLAMAN by MANN fibroblasts

FIG. 11. SDS-PAGE gel of purified LAMAN (lanes 4 and 7)

### EXAMPLES

### Example 1

Cloning of hLAMAN cDNA and Construction of Expression Vector

PCR and Cloning

The source of the PCR DNA template was a HepG2 cDNA library made from mRNA isolated from the HepG2 cell line (ATCC #HB-8065) using the Superscript Plasmid System manufactured by Gibco BRL, catalogue #18248-013. Two hundred ng of DNA from this library was amplified using Klentaq polymerase mix (Clontech, catalogue #84171-1) in a 50 ul reaction volume containing 0.2 mM dNTPs and 0.4 uM each of ICO1106 (5′ TCTGCGC TCGAGGCTGCCTGGACTCA, SEQ ID NO. 3) and ICO1107 (5′ TGCTAATCTAGAGGG CCCATCCCAGCAGAC, SEQ ID NO. 4). Two-cycle PCR was used with an initial denaturation step at 96° C. for 2 minutes followed by 30 cycles of 94° C. for 30 seconds and 65° C. for 4.5 minutes. A final extension of 10 minutes at 72° C. was used at the end to ensure that the extension products were filled out.

The PCR reaction mix was run out on a gel and a PCR product of 3038 bp was isolated. One twentieth of this material was re-amplified with the same two primers as described above, except that the temperature of the extension reaction was 68° C. The 3038 bp amplified fragment was gel isolated, digested with Xho I and Xba I and assembled into plasmid pAsaExp1 (FIG. 5 and 6) cut with EcoR I and Xba I together with oligos ICO1108 (5′AATTCGCCGCCATGGGCGCCTACGCGCGGGCTTCGGGGGTCTGCGC) and ICO1109 (5′TCGAGCGCAGACCCCCGAAGCCCG CGCGTAGGCGCCCATGGCGGCG) in a three part litigation (FIG. 2).

Sequencing

Seven plasmid clones from the ligations described above were sequenced with the Big dye terminator cycle sequencing kit from PE/ABI (catalogue #4314417). Two vector primers, ICO873 (5′ TGCAGCTTATAATGGTTACA, SEQ ID NO. 5) and ICO929 (5′ GGCACCTATTGGTCTTACTG, SEQ ID NO. 6) and five LAMAN-specific primers, ICO1110 (5′ AAGTCGTGCGAGACCTTGTG, SEQ ID NO. 7), ICO1111 (5′ CCGCCAAGGA ATCTGTGCTG, SEQ ID NO. 8), ICO1112 (5′ TACGAGCGCCTCAGCTACAA, SEQ ID NO. 9) and ICO1113 (5′ GCGGAAGGTGAATTGGATGG, SEQ ID NO. 10) and ICO1114 (5′ GCCTCAGGTGCC TACATCTT, SEQ ID NO. 11) were used. The results are summarized in Table 1.

Construction of the Correct, Full-Length LAMAN cDNA

As seen from Table 1, none of the seven cDNA clones match the published sequence (Nilssen et al. Hum.Mol.Genet. 6, 717-726 1997, revised version Accession #U60266.1). In addition to a number of random changes they all have the TA to AT changes at positions 1670 and 1671 in the plasmid sequence (SEQ ID NO: 1). This results in a valine to asparatic acid substitution in residue 186 of the LAMAN protein sequence, see SEQ ID NO. 12. The same valine to asparatic acid change is also reported by two other research groups (Liao et al. J.Biol.Chem. 271, 28348-28358, 1996 and Nebes et al. Biochem Biophys. Res. Commun. 200, 239-245, 1994). Hence, for the purpose of this study, a full-length LAMAN sequence was patched together from plasmid clones pLaman-50 and pLaman-70 using the unique SanD I site at position 2388 (FIGS. 2 and SEQ ID NO: 2).

A 1287 bp EcoR I-SanD I fragment from pLaman-50 and a 1779 bp SanD I-Xba I fragment from pLaman-70 were isolated and assembled together in plasmid pAsaExp1 (FIG. 5 and 6) cut with EcoR I and Xba I in a three part ligation (FIG. 2). The resulting plasmid is called pLamanExp1. The LAMAN insert in this plasmid was confirmed by sequencing and found to match the sequence (Nilssen et al. Hum.Mol.Genet. 6, 717-726, 1997 accession #U60266.1) except at amino acid position 186 as referred to in the plasmid sequence shown in SEQ ID NO: 2. The map of this plasmid is shown in FIG. 3.

TABLE 1
Summary table of sequencing results

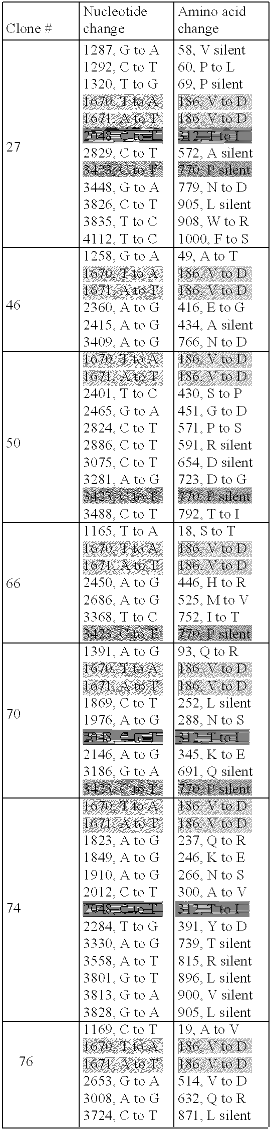

NOTE:
The clones are in plasmid pLamanExp1. The numbering is as shown in the plasmid sequence (SEQ ID NO:2). Identical changes between clones are highlighted in different colors. The reference sequence for the lysosomal alpha-mannosidase cDNA has the GenBank Accession number U60266.1 (1, revised).

Discussion

The inserts in all seven of the clones were confirmed to be LAMAN by sequence analysis. As seen in Table 1 a number of changes were found in all of the clones when compared to our reference sequence (1, revised version accession #U60266.1). Most of them are likely to be random PCR errors. Curiously, the TA to AT change at positions 1670 and 1671 resulting in a valine to asparatic acid substitution was common to all the clones. The two other sequences published for LAMAN (Nebs et al. Biochem. Biophys. Res. Commun. 200, 239-245, 1994, Liao et al. J.Biol.Chem. 271, 28348-28358, 1996) also report asparatic acid in that position. In the light of these observations a full-length correct sequence was put together with the help of the SanD I site at position 2388 as described under materials and methods. The sequence of the cDNA insert in this clone is identical to the reference sequence except for the valine to asparatic acid change mentioned above.

Evaluation and Conclusions

A combination of PCR amplification and incorporation of synthetic oligonucleotides assembled a full-length clone of LAMAN. It matches the sequence Accession #U2066.1, our reference sequence except in the valine to asparatic acid substitution alluded to earlier in this report. This LAMAN cDNA sequence also carries a C to A change at nucleotide position 1152 (see plasmid sequence in SEQ ID NO. 2). This was engineered to create a Xho I site for ease of manipulation and does not change the primary sequence of the LAMAN protein. It also contains a G to A change at position 3186 and a C to T change at position 3423 with no effect on the primary amino acid sequence of the LAMAN protein.

All seven of our clones had the valine to asparatic acid substitution in the 186th residue of the protein. Since the same substitution was also reported by two other groups (accession #s U68567 and U05572, refs. 2 and 3) the asparatic acid residue was left unchanged at that position. The reference sequence Accession #U60266.1 differs from the two other published sequences mentioned above at a few other positions. When compared to sequence Accession #U68567 (Liao et al. J.Biol.Chem. 271, 28348-28358, 1996) three other changes can be found. Curiously, the threonine to isoleucine change occurring at amino acid position 312 (Liao et al. J.Biol.Chem. 271, 28348-28358, 1996) was present in three of our clones (Table 1). None of our clones had either the 3 base-pairs deletion encoding the glutamine residue at position 342 or the G to T silent mutation in the leucine residue at position 753 found in (3, Accession #U05572). Three of our clones however, had a C to T silent change in the proline at position 770.

### Example 2

Transfection, Selection of Producer Cell and Preliminary Characterisation of Recombinant Human LAMAN

Materials and Methods:

Materials Used for Cell Culture

Media:

| Material | Company | Catalog # |
|---|---|---|
| Penicillin/Streptomycin | BioWhittaker | 17-602E |
| L-Glutamine, 200 mM | BioWhittaker | 17-605E |
| HT Supplement (100X) | Gibco BRL | 11067-030 |
| Ex-Cell 302 Serum-Free Media | JRH BioSciences | 14312-78P |

| | |
|---|---|
| Media for DG44.42 Cells: | |
| Ex-Cell 302 Serum-Free Medium | 500 ml |
| Penicillin/Streptomycin | 5 ml |
| L-Glutamine (4 mM final concentration) | 10 ml |
| 100X HT Supplement | 5 ml |
| Media for ASA Masterwell Cells: | |
| Ex-Cell 302 Serum-Free Medium | 500 ml |
| Penicillin/Streptomycin | 5 ml |
| L-Glutamine (4 mM final concentration) | 10 ml |
| 20 nM Methotrexate | 0.5 ml (20 μM MTX*) |

*Diluted from 20 mM Stock.

Preparation of 20 mM Methotrexate Stock:

Materials:

1. Methotrexate, U.S.P.: ICN catalogue #102299

2. WFI (Water for Injection)

3. 1.0N NaOH to adjust pH.

4. 0.2μm filter unit of appropriate size

Gel Electrophoresis:

Gel electrophoresis was done using the Novex system with Nupage Bis/Tris gels run at 200 volts for 1.2 hours. Staining was done with Coomassie brilliant blue R-250 according to the manufacturer's specifications. Total protein was measured by the Bradford method using the BioRad reagent (catalogue #500-006) and bovine serum albumin as standard.

LAMAN Enzyme Assay

Materials:

4× assay buffer consisting of:

250 μM p-Nitrophenyl-alpha-D-mannopyranoside

100 μg/mL BSA

200 mM Na Acetate (pH 4.5)

1.8 M Na₂CO₃

Procedure:

For screening purposes the assays were done in flat-bottomed ELISA plates. 50 μl of the 4× assay buffer was added to 150 μl of sample or an appropriate dilution of it (in 10 mM Tris pH 7.4 containing 150 mM NaCl). The plates were incubated overnight at room temperature, stopped with 100 μl of 1.8 M Na₂CO₃ and the absorbance recorded at 405 nm on a plate reader.

For determination of specific activity of the DEAE-purified samples, the assays were set up in tubes with all the volumes doubled. Incubations were at 37° C. for periods ranging from 3-60 minutes using 10-10,000 ng of enzyme. The samples were read on a spectrophotometer using a cuvette of 1 cm path length. Specific activity is defined as μmoles of p-Nitrophenyl-alpha-D-mannopyranoside hydrolysed per minute per mg protein.

Introduction

To be able to produce rhLAMAN the expression plasmid (pLamanExp1, FIG. 3, SEQ ID NO. 2) was cell type the following experiment was performed. To obtain the final clonal cell line expressing rhLAMAN, the expression plasmid pLamanExp1 (FIG. 3, SEQ ID NO. 2) was transfected into the host cell line DG44.42 (obtained from DG44 by limiting dilution). High LAMAN producing masterwells, as determined by LAMAN activity, were selected.

To further increase the yield, the cells from high LAMAN producing masterwells were subjected to amplification with methotrexate (MTX) as described by Gasser et al. Proc. Natl. Acad. Sci. U.S.A 79, 6522-6526. 1982. After amplification, the best producers were cloned twice by limiting dilution.

Transfection Conditions and Selection of High Producing Masterwells

1×10⁷ CHO DG44.42 cells were resuspended in 800 μl Hepes Buffered Electroporation Media with 1020 μg pLamanExp1 DNA (linearized with Ahd I)+120 μg salmon sperm DNA in a 0.4 cm electroporation cuvette. The cells were electroporated using a Bio-Rad Gene Pulser II set at 250 volts with 500 μFd capacitance. The contents of the cuvette were then transferred to a T-75 flask containing 20 ml of JRH Ex-Cell 302 Serum-Free Medium+HT (hypoxanthine and thymidine)+4 mM L-glutamine+100 units/ml penicillin+100 μg/ml streptomycin and then placed in a 37° C., 5% CO₂ incubator. 24 hours later the cells were counted, centrifuged and transferred to two T-flasks, one with 20 nM and the other with 50 nM MTX replacing the HT in the growth media described above. A media sample was saved and run in a LAMAN activity assay—see materials and methods. The culture showed activity and was plated in 96-well dishes as follows; 2 plates each at 500 and 1000 cells/well in 100 nM MTX and 5 plates each at 50, 100 and 200 cells/well in 20 and 50 nM MTX respectively. The wells were fed fresh media every 5-7 days. Four weeks post electroporation a LAMAN activity assay was done on media from wells containing 1 to 4 clones. The masterwells with the highest LAMAN activity were then expanded and run in a 6-day LAMAN activity assay. In a 6-day LAMAN assay cells were seeded at 1×10⁵ cells/ml in 5 ml media with MTX in a T-12.5 flask. Six days later the cells were counted. The cells were then centrifuged and the supernatant analyzed in the LAMAN activity assay. Results were normalized for cell number. The 12 best masterwells were then amplified.

Amplification

Six masterwells were amplified starting from 20 nM MTX and six starting from 50 nM MTX. Amplification was done by seeding cells at 1×10⁵ cells/ml in T-25 Falcon flasks, in 10 ml of media. The cells were split every 4-6 days and analyzed in a 6-day assay. The cells were then amplified to the next level (50, 100, 200 nM MTX) on the next split as well as the cells being maintained at previous MTX levels. No amplification occurred at 100 and 200 nM MTX. The highest producing masterwell at 50 nM MTX (20L-31) was selected for further subcloning.

1^{st} Round Limiting Dilution Cloning

20L-31 was spinner adapted before cloning. The cells were seeded at 1.25, 2.5, 5 and 10 cells/well in 100 μl media containing 10% conditioned media in a flat-bottom 96-well plate. They were fed with 100 μl fresh media every 5-7 days. The wells were scanned for single clones within 4 weeks and these cells were transferred to 24-well plates with 1 ml fresh media. Growing clones they were transferred to a T-25 flasks, expanded and run in the 6-day LAMAN assay. Twenty-seven clones were analyzed in this fashion. One clone was chosen on the basis of cell growth, viability and activity. The clone chosen, 20L-31.62, had twice the LAMAN activity as the corresponding masterwell.

2^{nd} Round Limiting Dilution Cloning

The first round clone 20L-31.62 was already spinner adapted and was taken through the same subsequent steps of limiting dilution cloning as described above. Twenty-seven clones were analyzed in a 6-day LAMAN assay and one clone was retained as the final clone on the basis of cell growth, viability and LAMAN activity. The clone chosen, 20L-31.62.5, was also twice as active as the masterwell. It was maintained in media containing 50 nM MTX. This clone has been designated “CHO-LAMAN 20L-31.62.5 020118”.

Characterization of rhLAMAN

The characterization of rhLAMAN for specific LAMAN activity was done with partially purified material from the first round clones.

Purification

A preliminary purification of rhLAMAN was attempted for the purposes of determining specific activity and characterise the product by gelelectrophoresis.

Briefly, 800 ml of conditioned media from the masterwell, 20L-31 (cultured for in T-flasks for 10 days) was dialyzed against 20 mM Tris, pH 7.6 and loaded on a 5 ml DEAE-Sepharose column (AP-Biotech, Catalogue #17-0709-01), equilibrated in the same buffer. The column was washed thoroughly with equilibration buffer containing 20 mM NaCl and then eluted in steps using 120 mM, 250 mM and 1 M NaCl. One ml fractions were collected and analyzed by Bradford protein assay and SDS-PAGE. The gel profile is shown in FIG. 5. The fractions are labeled serially according to the salt concentration used for elution. Two pools were collected. The content of each lane i described in table 2 below.

TABLE 2
Contents of 4-12% BisTris w MES/SDS reducing gels

| | | μg/ml by | total μg |
|---|---|---|---|
| Lane | Sample | Bradford | in lane |
| 1 | Marker | | |
| 2 | Start | 124 | 2.48 |
| 3 | Flow | 80 | 1.6 |
| | Through | | |
| 4 | 120.6 | 1827 | 36.54 |
| 5 | 120.7 | 1755 | 35.1 |
| 6 | 120.8 | 1250 | 25 |
| 7 | 120.9 | 963 | 19.26 |
| 8 | 120.10 | 619 | 12.38 |
| 9 | 250.1 | 477 | 9.54 |
| 10 | 250.2 | 361 | 7.22 |
| 11 | 250.3 | 303 | 6.06 |
| 12 | 250.4 | 334 | 6.68 |
| 13 | 250.5 | 460 | 9.2 |
| 14 | marker | | |
| 15 | 1.1 | 1023 | 20.46 |
| 16 | 1.2 | ND | ND |
| 17 | 1.3 | ND | ND |
| 18 | 1.4 | ND | ND |
| 19 | 1.5 | ND | ND |
| 20 | 1.6 | 1471 | 29.42 |
| 21 | 1.7 | ND | ND |
| 22 | 1.8 | ND | ND |
| 23 | 1.9 | ND | ND |
| 24 | 1.10 | ND | ND |

The samples correspond to the fractions collected. The fractions are numbered serially according to the salt concentration in the step gradient used (120 mM. 250 mM. 1 M). ND stands for not done.

The 120 mM pool (lanes 4-8 in FIG. 5) had a specific activity of 0.8 U/mg and 1.25 mg/ml total protein. The 250 mM pool (lanes 9-12 in FIG. 5) had a specific acivity of 1 U/mg and 0.48 mg/ml total protein.

Another purification was attempted starting with 1.2 L of dialyzed medium from 20L-31 grown in T-flasks applied to a 50 ml DEAE-Sepharose column. After washing the column in the same manner, rhLAMAN was eluted with a steep linear gradient of 20-120 mM NaCl. 5 ml fractions were collected and analyzed by SDS-PAGE. FIG. 6 shows the gel profile and the pooled fractions are marked. The content of each lane is described in table 3 below.

TABLE 3
Contents of 4-12% BisTris w MES/SDS reducing gel

| Lane | Fraction | mM NaCl |
|---|---|---|
| 1 | 10 μg of rhLaman from | 120 mM by Step elution |
| | purification shown in FIG. 5 | |
| 2 | Markers | |
| 3 | 5 | 40 |
| 4 | 6 | 40 |
| 5 | 10 | 50 |
| 6 | 11 | 50 |
| 7 | 12 | 50 |
| 8 | 16 | 60 |
| 9 | 18 | 70 |
| 10 | 60 | 140 |
| 11 | 62 | 140 |
| 12 | 63 | 150 |

The pooled material (25 ml) had a specific activity of 2 U/mg at a protein concentration of 1 mg/ml.

Conclusion

A LAMAN production cell line (CHO DG44.42 subclone 20L-31.62.5) was obtained after MTX amplification and two rounds of cloning. This clone has been designated “CHO-LAMAN 20L-31.62.5 020118” and deposited at the DSMZ (Deutsche Sammlung von Mikroorganismen und Zellkulturen GmbH, Mascheroder Weg 1b, 38124 Braunschweig, GERMANY) for the purposes of patent deposit according to the Budapest Treaty on Jun. 6, 2002.

The CHO cell-based expression system described here produces in excess of 20 mg/L rhLAMAN, which is secreted into the culture medium as a single-chain molecule. A preliminary purification was attempted. The purification resulted in a partially purified rhLAMAN preparation, see elution profile FIG. 5 and 6 that can be used for cellular uptake studies and further purification.

### Example 3

In Vitro Characterisation of rhASA

Materials and Methods:

Complete Medium

MEM Eagle-Earles BSS with

15% FCS

2 mM Glutamine

2× conc. Vitamins

2× conc. non-essential AA

2× conc. MEM-AA

1 mM Na-puruvate

TBS composition

10 mM Tris-HCl

150 mM NaCl

Adjust pH to 7.5

Alfa-Mannosidase activity assay protocol

4× assay buffer

16 mM para-Nitrophenyl-alpha-D-mannopyranoside

2 mg/ml BSA

in 0.4 M NaAc pH 4.5

Stop buffer

1.8 M Na₂CO₃

For measuring activity in cell supernatants, the assay was run in microtiter plate. For cell lysates, the assay was performed in Eppendorf tubes, see below.

Microtiter plate set-up

150 μl of sample at appropriate dilution (in TBS)

50 μl of 4× assay buffer

The plate is incubated at 37° C. for 30 minutes.

Stop by adding 100 μl stop buffer and read the plate at OD 405.

Eppendorf tube set-up

300 μl of sample at appropriate dilution (in TBS)

100 μl of 4× assay buffer

The tubes are incubated at 37° C. for 30 minutes to 2 hours.

Stop by adding 200 μl stop buffer, centrifuge the tubes (13 200 rpm, 10 minutes) and transfer the supernatant to a cuvette.

Measure at OD 405.

The LAMAN activity is then calculated using the millimolar extinction coefficient for the product in the formula:

(OD405×V)/(18.8×t×v)=U/ml

V=total volume for assay (ml)

t=incubation time (min)

v=sample volume (ml)

The millimolar extinction coefficient for para-nitrophenol=18.8 M⁻¹ cm⁻¹

1 U of LAMAN activity is defined as the amount of enzyme that liberates 1 μmol para-nitrophenol/min, 37° C., pH 4.5.

BCA Protein Determination Kit

Protein concentration was determined using the BCA Protein assay kit (no. 23225) from Pierce. This kit utilizes the principle of the reduction of Cu²⁺ to Cu⁺ by protein in an alkaline medium (the Biuret reaction). The Cu⁺ ions are then reacted with a reagent containing bicinchoninic acid resulting in a highly sensitive and selective colorimetric detection. The kit was performed according to the manual.

Microtiter plate set-up

10 μl of the BCA-Standards for a standard curve

10 μl of BSA reference in duplicate

10 μl of MilliQ water used as blank in at least triplicate

10 μl of sample or diluted sample in MilliQ water

200 μl of BCA assay buffer in all wells

Mix˜30 seconds

Incubate samples for 30 minutes at 37° C.

Measure the absorbance within 10 minutes at 562 nm and determine the concentrations from the standard curve.

Introduction

At an early stage, before the purification procedure was developed, the rhLAMAN was characterized in an in vitro cell system. For this purpose, LAMAN-containing supernatants from LAMAN-CHO cells were purified using a DEAE purification step. Briefly, LAMAN-containing supernatants from T flasks with the DG44.42 subclone 20L-31.62.5 were diafiltered and concentrated and then added to the column after equilibration with 10 mM Tric-HCL, pH 7.6. After loading, the column was washed with the same buffer and then with 10 mM Tris-HCl+0.02 M NaCl. 3 column volumes were used for each washing step. The bound proteins were eluted using a NaCl gradient ranging from 0.02 to 0.3 M NaCl. 5 ml fractions were collected. The fraction with the highest specific activity was purified approximately 100 times and the specific LAMAN activity was around 4 U/mg. This material was used in the cell systems described below.

1. Cellular Uptake of rhLAMAN by MANN Fibroblasts

Aim of the Study

To get in vitro proof of concept regarding cellular uptake of rhLAMAN in fibroblasts from individuals affected by alpha-mannosidosis (MANN fibroblasts).

Experimental Design

Fibroblasts from a patient with mannosidosis (MANN fibroblasts GM00654, purchased from Coriell Cell Repository, USA) were grown confluent in 6-well plates in complete medium. Cells were washed with PBS and rhLAMAN (diafiltrated, concentrated and DEAE-purified, see purification part for details) was added (0-200 mU/ml)±5 mM Mannose-6-Phosphate (M-6-P) or Glucose-6-Phosphate (G-6-P, control) in complete medium. The specific LAMAN activity of the added enzyme was 4 U/mg total protein. The cells were left for 40 hours and then harvested; medium was removed, centrifuged and buffer was changed to TBS using PD10 columns. Cells were washed with PBS and trypsinated, washed with TBS and then lysed with 0.5% Triton X-100 in TBS. The lysed cells were centrifuged (13 200 rpm, 10 minutes) and lysate was collected. LAMAN activity and protein concentration were measured in all samples.

For details on the LAMAN activity assay and complete medium composition, see Materials and methods. Protein determination was performed in micro-titer plate according to our internal SOP. See Materials and methods for a brief description of the method.

Results and Discussion

The MANN fibroblasts had a background LAMAN activity of 1-1.5 mU/mg total protein. DEAE-purified rhLAMAN gave a maximal cellular load of 37 mU/mg total protein (after addition of approximately 75 mU/ml for 40 hours), see FIG. 7. Addition of M-6-P inhibited 90% of the cellular uptake and there was no inhibition using G-6-P. This suggests that the enzyme is taken up mainly by the M-6-P receptor. Results are shown in FIG. 8 and 9.

The experiment was repeated with a purer rhLAMAN with a higher specific enzymatic activity.

Experimental Design

Fibroblasts from a mannosidosis patient (MANN-fibroblasts GM00654, purchased from Coriell Cell Repository, USA) were grown confluent in 6-well plates in complete medium. Cells were washed with PBS and purified rhLAMAN (DEAE Sepharose and gel filtration) with a specific activity of 15 U/mg and a purity of more than 90% (See purification part for details) was added (0-200 mU/ml)±5 mM Mannose-6-Phosphate (M-6-P) or Glucose-6-Phosphate (G-6-P, control) in complete medium. The cells were incubated for 66 hours and then harvested; medium was removed, centrifuged and diluted 100 to 200 times in TBS. Cells were washed with PBS, trypsinated, washed with TBS and then lysed in TBS by repeated freeze thawing. The lysed cells were centrifuged (13 200 rpm, 10 minutes) and lysate was collected. LAMAN activity and protein concentration was measured in all samples.

Results and Discussion

A more pure rhLAMAN with higher specific activity resulted in a maximal cellular load of 90 mU/mg total protein after addition of 200 mU/ml for 66 hours (FIG. 9). Almost no cellular uptake of rhLAMAN was obtained after addition of 5 mM M-6-P compared to the control cells incubated with 5 mM G-6-P (FIG. 10). This indicates that the enzyme is taken up by the M-6-P receptor.

### Example 4

Small-Scale Cultivation of Producer Cell

Cell Line and Culture Medium

A DHFR minus Chinese Hamster Ovary (CHO) cell line, DG44.42 subclone 20L-31.62.5, expressing rhLAMAN, is routinely cultivated in modified EX-CELL 302 CHO serum-free medium without phenol red (JHR Biosciences Europe, UK). The medium contains 1.6 g/L sodium bicarbonate, 4 mM HEPES and 0.1% Pluronic®F-68, and is supplemented with 4 mM L-Glutamine and 3.4 g/L D-(+)-Glucose. The pH of the medium is 7.0-7.4. Selection is maintained by using 20 nM methotrexate (MTX), a competitive inhibitor of DHFR. 1 mL of an 20 μM MTX working solution is added to 1 L of complete medium.

No antibiotics are used. Samples for mycoplasma test are obtained periodically and analysed by the Mycoplasma Laboratory at the National Veterinary Institute, Uppsala, Sweden. All samples tested were found to be negative for mycoplasma.

T-Flasks

T-flasks (175 cm²) with 60 mL medium are seeded with 2.5-3×10⁵ cells/mL and incubated at 37° C. and 5% CO₂ in a humidified incubator for 72-96 hours. After 3-4 days, a cell concentration of 1-1.2×10⁶ cells/mL is reached. The T-flasks are gently tapped to break any cell aggregates. The cell suspensions are centrifuged (110 g, 8 min) in sterile tubes in a swing-out centrifuge (Eppendorf) at 20° C. The collected supernatants are filtered (0.45 μm) and passed on to the purification process or stored frozen (−20° C.) for later use. Fresh and filtered supernatants from T-flasks hold a LAMAN activity of 30 mU/mL. The cell pellets are resuspended in fresh medium at RT to a suitable cell density and passaged to new T-flasks cultures.

Counting of viable and dead cells is performed by using a haemocytometer and the trypan blue exclusion technique. Stored cells are frozen in liquid nitrogen in a working cell bank. New vials of cells are thawed regularly, at least every fifth month, and the LAMAN production is checked every week during the cultivation process.

### Example 5

Partially Developed Purification Scheme, Formulation, Filling and Lyophilization

A purification process for rhLAMAN in 20-200 ml scale is developed for scale-up to large scale production. The quality and purity of the final product (rhLAMAN) is very high and according to the specifications (approved for clinical trials). rhLAMAN is expressed in CHO cells and excreted into the media as a 260 kDa precursor in a dimer form (each subunit is 130 kDa). The process includes a capture step, 1-2 intermediate purification steps, 1 polishing step, 1-2 virus removal steps and 1 formulation step. 1 or more buffer exchange steps are also included (dia-filtration). The small-scale process is transferred to intermediate and finally large-scale production.

For the first proof of concept studies in vitro and in vivo, a reduced purification procedure has been used (including steps 1-4+8), resulting in >90% purity and a specific activity of the LAMAN precursor form of 15-20 U/mg.

Experimental design: Several different chromatography gels are tested and performance of the different steps will be analysed by a battery of analytical methods described briefly below:

| | |
|---|---|
| Enzyme activity: | Alpha-mannosidase assay |
| Total protein concentration: | BCA analysis |
| rhLAMAN concentration: | rhLAMAN ELISA |
| Purity: | HPLC and SDS-PAGE |
| Identity: | HPLC and western blot rhLAMAN |
| HCP proteins: | ELISA |
| Endotoxin level: | Outsource to contract Lab |

Outline of Purification Process in 20 ml Column Scale

Step 1: Concentration/Diafiltration

Media produced in T-500 flasks (0.06U/ml) with expressed rhLAMAN is concentrated approximately 10× for using Tangential flow filtration (TFF) against a Pellicon Biomax polysulphone filter with 100 kDa cut off (Millipore).

Example: 0.5-1.0 litre spinner produced media is concentrated using TFF against a 50 cm² Biomax 100 kDa filter to 50-100 ml. Transmembrane pressure (TMP) is 25 (Pin=30 psi; Pout=20 psi).

After that, diafiltration against 4-6 volumes of 20 mM Tris-HCl, pH 7.6 with TMP of 25 is applied. Specific activity of concentrated sample is 0.5-1.5 U/mg. Yield 70-90%

Step 2: Capture Step—DEAE Sepharose FF

Concentrated sample from step 1 is applied on a 20 ml DEAE sepharose packed in a 16 mm diameter column (Pharmacia XK 16) equilibrated with 20 mM Tris-HCl pH 7.6 (referred to as: standard buffer). Flow rate is 3 ml/min. Protein bound to the DEAE gel is then washed with 2-4 column volumes (CV) of standard buffer followed by 2-4 CV's of 30 mM NaCl in standard buffer.

rhLAMAN is eluted with a linear gradient from 30-300 mM NaCl in standard buffer for 20 minutes. Alternatively for the large scale production: Elution is achieved by applying step-wise elution, 75-150 mM NaCl in standard buffer. The DEAE gel is washed with 2-4 CV's of 0.5 M NaCl in standard buffer followed by 2-4 CV's of 1.0 M NaOH (contact time 40-60 minutes). Fractions containing rhLAMAN activity are pooled (specific activity: 2-5 U/mg) and used for further purification. Yield 70-90%.

Step 3: Concentration

Small scale: The rhLAMAN pool from step 2 is applied in an Amicon Centricon Plus-80 centrifugation filter with a 30 kDa cut off (Millipore). Sample is concentrated at 3000 g to a volume of 1-3 ml and a concentration of approximately 5-10 mg/ml. Yield 90-100%. Medium to large scale: Concentration using TFF (membrane with a 100 kDa cut of) is applied.

Step 4: Intermediate Step 1—Gel Filtration Sephacryl S-300 HR (Amhersam Bioscienses)

Sephacryl S-300 HR (26/60) is equilibrated with a standard buffer+0.15 M NaCl. Concentrated LAMAN pool from step 3 is applied to the column and run at 1.3 ml/min. Protein peak with activity is pooled and used in step 5. Yield is 80-90% and purity>90%. As an alternative, Superdex-200 (Amhersam Bioscienses) is used.

Step 5: Intermediate Step 2

1. Hydrophobic interaction chromatography: butyl, phenyl or octyl sepharose FF. Sample pool from step 4 is mixed 1:1 with 2.0 M Na₂SO₄ and applied on a 20 ml HIC column (butyl-, phenyl- or octyl-sepharose) packed in a 16 mm diameter column (Pharmacia XK 16) equilibrated with standard buffer+1.0 M Na2SO4. Flow rate is 3-4 ml/min. Column is washed with 2-4 CV's of the same buffer. rhLAMAN is eluted with standard buffer and fractions containing activity are pooled and used for further purification.

2. Macroprep, Ceramic hydroxyapatite type I or II (BioRAd) Brief description: Equlibrate column (Gel volume=20 ml) with 4-6 CV's of 10 mM Sodium phosphate H 7.6 (Buffer A). Flow rate 2-5 ml/min. Buffer exchange sample from step 3 with TFF to Buffer A. Load sample from step 3 on column. Wash with 2-4 CV's of Buffer A. Elute with buffer A containing 100 mM NaCl. Collect peak containing rhLAMAN activity.

3. Source 15 Q—Anionexchanger Equilibrate column with 4 CV's of 200 mM Tris-HCl pH 7.6. Change to 5 CV's of 20 mM Tris-HCl pH 7.6 (standard buffer). Flow rate: 2-5 ml/min. Load sample from step 4 (should be in standard buffer before application) onto column. Wash with 2-4 CV's of standard buffer. Apply a shallow gradient from 0 to 100% of 1 M NaCl in standard buffer (flow rate 2 ml/min, gradient time 50 minutes). Collect fractions containing rhLAMAN activity.

4. Source 15 S—Cation Exchanger Should run in acidic pH. Equilibration buffer=20 mM Sodium Acetate pH 4.5. Elute with a NaCl (increasing salt concentration) or pH (increasing pH) gradient.

Step 6: Polishing Step

1. Macro prep, Ceramic hydroxyapatite type II, 40 um. Description of parameters: see Step 5.

2. Source 15 Q—anion exchanger Description of parameters: see Step 5.

3. Source 15 S—cation exchanger Description of parameters: see Step 5.

4. Affinity chromatography

Step 7: Virus Filtration Step

Virus filtration will be performed on the product pool from step 5 using a 0.1 micron sterile filter followed by a DV 20 nanofilter from Pall with an applied constant pressure of 20-50 psi.

As an alternative, 1% of Tween 20 or 80 could be applied to the supertenant (contact time 30-60 minutes) before the first concentration and diafiltration step (step 1)

Step 8: Diafiltration/Formulation Step

For small scale: The rhLAMAN pool from step 4 is applied in an Amicon Centricon Plus-80 centrifugation filter with a 30 or 100 kDa cut off. Samlpe is concentrated at 3000 g to a volume of 1-3 ml and a concentration of approximately 5-10 mg/ml. Re-suspension with 15 ml of desired buffer is performed twice and centrifugation procedure repeated. Yield 90-100%.

For medium to large scale: Tangential flow filtration (TFF) against a Millipore Biomax 30 or 100 kDa screen type A against 5-10× Volumes of formulation buffer is performed. Two formulation buffers are tested:

Formulation buffer 1.

| | |
|---|---|
| Na₂HPO₄ | 3.50-3.90 mM |
| NaH₂PO₄ | 0.0-0.5 mM |
| Glycine | 25-30 mM |
| Mannitol | 220-270 mM |

Water for injection (WFI)

Formulation buffer 2.

| | |
|---|---|
| Sodium Citrate | 4.0-5.0 mM |
| Citric Acid | 0.3-0.8 mM |
| Mannitol | 200-250 mM |
| Tween 80 | 3.0-5.0 mM |

Water for injection (WFI)

The pH and osmolality in both Formulation buffers are balanced to 7.7±0.2 and 300±50 mOsm/kg respectively. Final protein concentration is according to the specification (>5 mg/ml).

Step 9: Formulation, Filling and Freeze-Drying

Formulation and Dosage Form

In the development of dosage form the stability of rhLAMAN is an important factor to consider. The development process starts with an aqueous solution and will, most likely, end up as a freeze-dried product. Two different formulations are tested: Formulation buffer 1 and Formulation buffer 6, see Step 7.

Both these formulations are known to stabilize proteins in aqueous solutions as well as in freeze-dried powders. The pH and osmolality in both Formulation buffers will be balanced to 7.7±0.2 and 300±50 mOsm/kg respectively. Final protein concentration should be according to the specification and in the range of 5-20 mg/ml.

A freeze-dried product of rhLAMAN will be produced at a production unit according to EU GMP practice. The filling and freeze-drying will be performed in a room classified as Class A. During production the filling zone is monitored with particle count and settle plates. The personnel are regularly trained according to EU GMP and monitored after each production with glove prints. The sterility of equipment and materials are secured by validated sterilization procedures.

Filling

The bulk drug substance of rhLAMAN are aseptically filled in sterile type I glass vials.

Freeze-Drying

The vials are freeze-dried with freeze-drying cycles specifically developed for rhLAMAN in the two different formulations described above. Nitrogen gas is filled into the vials in the end of the cycle and eventually closed with stoppers and capped. The batch is finally analyzed and released according to the specification.

Results From Small Scale Purification Process (20 ml Column Scale) Utilizing Steps 1-4+8 Described Above

Results from purification of rhLAMAN for in vivo and in vitro proof of concept are presented below:

| | |
|---|---|
| Overall yield: | 35-50% (based on LAMAN activity) |
| Purity: | >90% according to SDS-PAGE (130, 70 and 55 kDa |
| | form, see FIG. 12) |
| Specific activity: | 15-20 U/mg |
| Protein conc.: | 6.5-8.5 mg/ml |

Mainly 130 kDa precursor form, but also 70 and 55 kDa partly processed
forms (seen on Western blot and SDS-PAGE under reducing conditions)
Fragmentation occurs at TFF and DEAE steps and seems to be induced
non-enzymatically (protein very sensitive to fragmentation). This
fragmentation has earlier been described in the literature (Ref. Berg,
T. et al. (2001) Mol. Genet. Metab. 73, 18-29.)
Activation of the purified LAMAN precursor form has been noted upon
cell or serum contact up to 300% resulting in a specific activity of
40-60 U/mg.

References

Aebischer P, Goddard M, Signore A P, Timpson R L. 1994. Functional recovery in hemiparkinsonian primates transplanted with polymer-encapsulated PC12 cells. Exp Neurol 126:151-158

Aebischer P, Schluep M, Deglon N, Joseph J M, Hirt L, Heyd B, Goddard M, Hammang J P, Zurn A D, Kato A C, Regli F, Baetge E E. 1996. Intrathecal delivery of CNTF using encapsulated genetically modified xenogeneic cells in amyotrophic lateral sclerosis patients. Nat Med 2:696-699

Austin J, McAfee D, Armstrong D, O'Rourke M, Shearer L, Bachhawat B. 1964. Abnormal sulphatase activities in two human diseases (metachromatic leucodystrophy and gargoylism). Biochem J 93:15C-17C

Barth M L, Fensom A, Harris A. 1995. Identification of seven novel mutations associated with metachromatic leukodystrophy. Hum Mutat 6(2):170-176

Bayever E, Ladisch S, Philippart M, Brill N, Nuwer M, Sparkes R S, Feig S A. 1985. Bone-marrow transplantation for metachromatic leucodystrophy. Lancet 2(8453):471-473

Bradley R T, Manowitz P. 1988. Electrochemical determination of lysosomal alpha-mannosidasectivity using high-performance liquid chromatography. Anal Biochem 173(1):33-38

Braulke T, Hille A, Huttner W B, Hasilik A, von Figura K. 1987. Sulfated oligosaccharides in human lysosomal enzymes. Biochem Biophys Res Commun 143(1):178-185

Braulke T, Tippmer S, Chao H J, von Figura K. 1990. Insulin-like growth factors I and II stimulate endocytosis but do not affect sorting of lysosomal enzymes in human fibroblasts. J Biol Chem 265(12):6650-6655

Deglon N, Heyd B, Tan S A, Joseph J M, Zurn A D, Aebischer P. 1996. Central nervous system delivery of recombinant ciliary neurotrophic factor by polymer encapsulated differentiated C2C12 myoblasts. Hum Gene Ther 7(17):2135-2146

Dierks T, Schmidt B, von Figura K. 1997. Conversion of cysteine to formylglycine: a protein modification in the endoplasmic reticulum. Proc Natl Acad Sci USA 94(22):11963-11968

Draghia R, Letourneur F, Drugan C, Manicom J, Blanchot C, Kahn A, Poenaru L, Caillaud C. 1997. Metachromatic leukodystrophy: identification of the first deletion in exon 1 and of nine novel point mutations in the lysosomal alpha-mannosidase gene. Hum Mutat 9(3):234-242

Draper R K, Fiskum G M, Edmond J. 1976. Purification, molecular weight, amino acid, and subunit composition of lysosomal alpha-mannosidase from human liver. Arch Biochem Biophys 177(2):525-538

Dubois G, Turpin J C, Baumann N. 1975. Arylsulfatases isoenzymes in metachromatic leucodystrophy/detection of a new variant by electrophoresis improvement of quantitative assay. Biomedicine 23(3):116-119

Eto Y, Tokoro T, Liebaers I, Vamos E. 1982. Biochemical characterization of neonatal multiple sulfatase deficient (MSD) disorder cultured skin fibroblasts. Biochem Biophys Res Commun 106(2):429-34

Farooqui A A. 1976. Purification and properties of human placenta arylsulphatase A. Arch Int Physiol Biochim 84(3):479-492

Farrell D F, MacMartin M P, Clark A F. 1979. Multiple molecular forms of lysosomal alpha-mannosidase in different forms of metachromatic leukodystrophy (alpha-mannosidosis). Neurology 29(1):16-20

Fujii T, Kobayashi T, Honke K, Gasa S, Ishikawa M, Shimizu T, Makita A. 1992. Proteolytic processing of human lysosomal lysosomal alpha-mannosidase. Biochim Biophys Acta 1122(1):93-98

Gieselmann V, Polten A, Kreysing J, Kappler J, Fluharty A, von Figura K. 1991. Molecular genetics of metachromatic leukodystrophy. Dev Neurosci 13:222-227

Gieselmann V, Zlotogora J, Harris A, Wenger D A, Morris C P. 1994. Molecular genetics of metachromatic leukodystrophy. Hum Mutat 4:233-42

Gieselmann V, Matzner U, Hess B, Lullmann-Rauch R, Coenen R, Hartmann D, D'Hooge R, DeDeyn P, Nagels G. 1998. Metachromatic leukodystrophy: molecular genetics and an animal model. J Inherit Metab Dis 21:564-574

Gniot-Szulzycka J. 1974. Some properties of highly purified arylsulphatase A from human placenta. Acta Biochim Pol 21(3):247-254

Gustavson K H, Hagberg B. 1971. The incidence and genetics of metachromatic leucodystrophy in northern Sweden. Acta Paediatr Scand 60(5):585-590

Hess B, Saftig P, Hartmann D, Coenen R, Lullmann-Rauch R, Goebel H H, Evers M, von Figura K, D'Hooge R, Nagels G, De Deyn P, Peters C, Gieselmann V. 1996. Phenotype of lysosomal alpha-mannosidase-deficient mice: relationship to human metachromatic leukodystrophy. Proc Natl Acad Sci USA 93(25):14821-14826

Hickey W F, Kimura H. 1988. Perivascular microglial cells of the CNS are bone marrow-derived and present antigen in vivo. Science 239(4837):290-2

Hickey W F, Hsu B L, Kimura H. 1991. T-lymphocyte entry into the central nervous system. J Neurosci Res 28(2):254-60

Inoue H, Seyama Y, Yamashita S. 1986. Specific determination of lysosomal alpha-mannosidase activity. Experientia 15;42(1):33-35

James G T. 1979. Essential arginine residues in human liver lysosomal alpha-mannosidase. Arch Biochem Biophys 197(1):57-62

James G T, Thach A B, Klassen L, Austin J H Studies in metachromatic leukodystrophy: XV. 1985. Purification of normal and mutant lysosomal alpha-mannosidase from human liver. Life Sci 37(25):2365-2371

Joss V, Rogers T R, Hugh-Jones K, Beilby B, Joshi R, Williamson S, Foroozanfar N, Riches P, Turner M, Benson P D, Hobbs J R. 1982 Exp Hematol 10, 52

Kelly B M, Yu C Z, Chang P L. 1989. Presence of a lysosomal enzyme, arylsulfatase-A, in the prelysosome-endosome compartments of human cultured fibroblasts. Eur J Cell Biol 48(1):71-78

Klein M A, Frigg R, Flechsig E, Raeber A J, Kalinke U, Bluethmann H, Bootz F, Suter M, Zinkernagel R M, Aguzzi A. 1997. A crucial role for B cells in neuroinvasive scrapie. Nature 390(6661):687-690

Kreysing J, Polten A, Hess B, von Figura K, Menz K, Steiner F, Gieselmann V. 1994. Structure of the mouse lysosomal alpha-mannosidase gene and cDNA. Genomics 19(2):249-256

Kreysing J, von Figura K, Gieselmann V. 1990. Structure of the lysosomal alpha-mannosidase gene. Eur J Biochem 1990. Eur J Biochem. 191(3): 627-631

Krivit W, Shapiro E, Kennedy W, Lipton M, Lockman L, Smith S, Summers C G, Wenger D A, Tsai M Y, Ramsay N K, et al. 1990. Treatment of late infantile metachromatic leukodystrophy by bone marrow transplantation. N Engl J Med 322:28-32

Krivit W, Shapiro E, Hoogerbrugge P M, Moser H W. 1992. State of the art review. Bone marrow transplantation treatment for storage diseases. Keystone. Jan. 23, 1992. Bone Marrow Transplant 10 Suppl 1:87-96

Laidler P M, Waheed A, Van Etten R L. 1985. Structural and immunochemical characterization of human urine lysosomal alpha-mannosidase purified by affinity chromatography. Biochim Biophys Acta 827(1):73-83

Lee G D, van Etten R L. 1975. Evidence of an essential histidine residue in rabbit liver aryl sulfatase A. Arch Biochem Biophys 171(2):424-434

Luijten JAFM, Van Der Heijden MCM, Rijksen G, Staal GEJ. 1978. Purification and characterization of lysosomal alpha-mannosidase from human urine. J Mol Med. 1978, 3, 213

Manowitz P, Fine L V, Nora R, Chokroverty S, Nathan P E, Fazzaro J M. 1988. A new electrophoretic variant of lysosomal alpha-mannosidase. Biochem Med Metab Biol 39(1):117-120

Mould D L, Dawson A M, Rennie J C. 1970. Very early replication of scrapie in lymphocytic tissue. Nature 228(273):779-780

Ohashi T, Watabe K, Sato Y, Saito I, Barranger J A, Matalon R, Eto Y. 1996. Gene therapy for metachromatic leukodystrophy. Acta Paediatr Jpn 38(2):193-201

Oya Y, Nakayasu H, Fujita N, Suzuki K, Suzuki K. 1998. Pathological study of mice with total deficiency of sphingolipid activator proteins (SAP knockout mice). Acta Neuropathol (Berl) 96(1):29-40

Poretz R D, Yang R S, Canas B, Lackland H, Stein S, Manowitz P. 1992. The structural basis for the electrophoretic isoforms of normal and variant human platelet arylsulphatase A. Biochem J 287 (Pt 3):979-983

Porter M T, Fluharty A L, Kihara H. 1971. Correction of abnormal cerebroside sulfate metabolism in cultured metachromatic leukodystrophy fibroblasts. Science 1971,172, 1263-1265

Sangalli A, Taveggia C, Salviati A, Wrabetz L, Bordignon C, Severini G M. 1998. Transduced fibroblasts and metachromatic leukodystrophy lymphocytes transfer lysosomal alpha-mannosidase to myelinating glia and deficient cells in vitro. Hum Gene Ther 9(14):2111-2119

Sarafian T A, Tsay K K, Jackson W E, Fluharty A L, Kihara H. 1985. Studies on the charge isomers of lysosomal alpha-mannosidase. Biochem Med 33(3):372-380

Schmidt B, Selmer T, Ingendoh A, von Figura K. 1995. A novel amino acid modification in sulfatases that is defective in multiple sulfatase deficiency. Cell 82(2):271-278

Selmer T, Hallmann A, Schmidt B, Sumper M, von Figura K. 1996. The evolutionary conservation of a novel protein modification, the conversion of cysteine to serinesemialdehyde in arylsulfatase from Volvox carteri. Eur J Biochem 238:341-345

Shapira E, Nadler H L. 1975. Purification and some properties of soluble human liver arylsulfatases. Arch Biochem Biophys 170(1):179-187

Shapiro E G, Lockman L A, Balthazor M, Krivit W. 1995. Neuropsychological outcomes of several storage diseases with and without bone marrow transplantation. J Inherit Metab Dis 18:413-429

Stein C, Gieselmann V, Kreysing J, Schmidt B, Pohlmann R, Waheed A, Meyer H E, O'Brien J S, von Figura K. 1989. Cloning and expression of human lysosomal alpha-mannosidase. J Biol Chem 264:1252-1259

Stevens R L, Fluharty A L, Skokut M H, Kihara H. 1975. Purification and properties of arylsulfatase. A from human urine. J Biol Chem 250(7):2495-2501

Stevens R L, Fluharty A L, Killgrove A R, Kihara H. 1976. Microheterogeneity of lysosomal alpha-mannosidase from human tissues. Biochim Biophys Acta 445(3):661-671

von Figura K, Steckel F, Conary J, Hasilik A, Shaw E. 1986. Heterogeneity in late-onset metachromatic leukodystrophy. Effect of inhibitors of cysteine proteinases. Am J Hum Genet, 39, 371-382

Waheed A, Hasilik A, von Figura K. 1982. Synthesis and processing of lysosomal alpha-mannosidase in human skin fibroblasts. Hoppe Seylers Z Physiol Chem 363(4):425-430

Waheed A, van Etten R L. 1985. Phosphorylation and sulfation of lysosomal alpha-mannosidase accompanies biosynthesis of the enzyme in normal and carcinoma cell lines. Biochim Biophys Acta 847(1):53-61

1. A cell capable of producing recombinant human LAMAN (rhLAMAN), said cell comprising the 3066 basepair EcoRI-XbaI fragment of a human cDNA which codes for a human LAMAN protein in which the position corresponding to position 186 of the full length hLAMAN protein is Aspartic acid (Asp, D).

2. A cell according to claim 1 comprising a DNA sequence which codes for the amino acid sequence shown in SEQ ID NO 12.

3. A cell according to claim 1 obtained by insertion of a DNA fragment comprising the 3066 basepair EcoRI-XbaI fragment of the DNA fragment having the sequence shown in SEQ ID NO 2.

4. A cell according to any of claims 1-3 obtained by use of the expression plasmid pLamanExp1 having SEQ ID NO. 2.

5. A cell according to any of claims 1-4 obtained by transfection of a non-human mammalian cell line.

6. A cell according to claim 5 obtained by transfection of chinese hamster ovary (CHO) cells.

7. A cell according to any of claims 1-6 obtained by the culture of the human LAMAN production cell line CHO DG44.42 subclone 20L-31.62.5 which has been deposited at the DSMZ for the purposes of patent deposit according to the Budapest Treaty on Jun. 6, 2002.

8. A method for the preparation of recombinant human LAMAN, the method comprising
a) introducing, into a suitable vector, a nucleic acid fragment comprising a DNA fragment which codes for the amino acid sequence shown SEQ ID NO 12;
b) transforming a cell with the vector obtained in step a);
c) culturing the transformed host cell under conditions facilitating expression of the nucleic acid sequence;
d) recovering the expression product from the culture.

9. A method according to claim 8 further comprising a fermentation step.

10. A method according to claim 8 or 9 further comprising at least one purification step.

11. A method according to any of claims 8-10, wherein the nucleic acid fragment comprises the 3066 basepair EcoRI-XbaI fragment of the DNA fragment shown in SEQ ID NO 2.

12. An expression plasmid pLamanExp1 having the sequence shown in SEQ ID NO. 2.

13. A rhLAMAN produced by the method of any of claims 8-11.

14. A method of treating a person having alfa mannosidosis, the method comprising administering a rhLAMAN according to claim 13.


SEQUENCE LISTING (12 sequences)

SEQ ID NO: 1 - DNA, 6591 bp, Artificial Sequence
  Expression plasmid pAsaExp1
agatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcgccaccatgggggcaccgcggtccctcctcctggccctggctgctggcctggccgttgcacgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgcctgagggcccctcggctggcctgggcatgtgatggctcctcactggtctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccaccttagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaattcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 2 - DNA, 8079 bp, Artificial Sequence
  Expression plasmid pLamanExp1
agatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcgccgccatgggcgcctacgcgcgggcttcgggggtctgcgctcgaggctgcctggactcagcaggcccctggaccatgtcccgcgccctgcggccaccgctcccgcctctctgctttttccttttgttgctggcggctgccggtgctcgggccgggggatacgagacatgccccacagtgcagccgaacatgctgaacgtgcacctgctgcctcacacacatgatgacgtgggctggctcaaaaccgtggaccagtacttttatggaatcaagaatgacatccagcacgccggtgtgcagtacatcctggactcggtcatctctgccttgctggcagatcccacccgtcgcttcatttacgtggagattgccttcttctcccgttggtggcaccagcagacaaatgccacacaggaagtcgtgcgagaccttgtgcgccaggggcgcctggagttcgccaatggtggctgggtgatgaacgatgaggcagccacccactacggtgccatcgtggaccagatgacacttgggctgcgctttctggaggacacatttggcaatgatgggcgaccccgtgtggcctggcacattgaccccttcggccactctcgggagcaggcctcgctgtttgcgcagatgggcttcgacggcttcttctttgggcgccttgattatcaagataagtgggtacggatgcagaagctggagatggagcaggtgtggcgggccagcaccagcctgaagcccccgaccgcggacctcttcactggtgtgcttcccaatggttacaacccgccaaggaatctgtgctgggatgtgctgtgtgtcgatcagccgctggtggaggaccctcgcagccccgagtacaacgccaaggagctggtcgattacttcctaaatgtggccactgcccagggccggtattaccgcaccaaccacactgtgatgaccatgggctcggacttccaatatgagaatgccaacatgtggttcaagaaccttgacaagctcatccggctggtaaatgcgcagcaggcaaaaggaagcagtgtccatgttctctactccacccccgcttgttacctctgggagctgaacaaggccaacctcacctggtcagtgaaacatgacgacttcttcccttacgcggatggcccccaccagttctggaccggttacttttccagtcggccggccctcaaacgctacgagcgcctcagctacaacttcctgcaggtgtgcaaccagctggaggcgctggtgggcctggcggccaacgtgggaccctatggctccggagacagtgcacccctcaatgaggcgatggctgtgctccagcatcacgacgccgtcagcggcacctcccgccagcacgtggccaacgactacgcgcgccagcttgcggcaggctgggggccttgcgaggttcttctgagcaacgcgctggcgcggctcagaggcttcaaagatcacttcaccttttgccaacagctaaacatcagcatctgcccgctcagccagacggcggcgcgcttccaggtcatcgtttataatcccctggggcggaaggtgaattggatggtacggctgccggtcagcgaaggcgttttcgttgtgaaggaccccaatggcaggacagtgcccagcgatgtggtaatatttcccagctcagacagccaggcgcaccctccggagctgctgttctcagcctcactgcccgccctgggcttcagcacctattcagtagcccaggtgcctcgctggaagccccaggcccgcgcaccacagcccatccccagaagatcctggtcccctgctttaaccatcgaaaatgagcacatccgggcaacgtttgatcctgacacagggctgttgatggagattatgaacatgaatcagcaactcctgctgcctgttcgccagaccttcttctggtacaacgccagtataggtgacaacgaaagtgaccaggcctcaggtgcctacatcttcagacccaaccaacagaaaccgctgcctgtgagccgctgggctcagatccacctggtgaagacacccttggtgcaggaggtgcaccagaacttctcagcttggtgttcccaggtggttcgcctgtacccaggacagcggcacctggagctagagtggtcggtggggccgatacctgtgggcgacacctgggggaaggaggtcatcagccgttttgacacaccgctggagacaaagggacgcttctacacagacagcaatggccgggagatcctggagaggaggcgggattatcgacccacctggaaactgaaccagacggagcccgtggcaggaaactactatccagtcaacacccggatttacatcacggatggaaacatgcagctgactgtgctgactgaccgctcccaggggggcagcagcctgagagatggctcgctggagctcatggtgcaccgaaggctgctgaaggacgatggacgcggagtatcggagccactaatggagaacgggtcgggggcgtgggtgcgagggcgccacctggtgctgctggacacagcccaggctgcagccgccggacaccggctcctggcggagcaggaggtcctggcccctcaggtggtgctggccccgggtggcggcgccgcctacaatctcggggctcctccgcgcacgcagttctcagggctgcgcagggacctgccgccctcggtgcacctgctcacgctggccagctggggccccgaaatggtgctgctgcgcttggagcaccagtttgccgtaggagaggattccggacgtaacctgagcgcccccgttaccttgaacttgagggacctgttctccaccttcaccatcacccgcctgcaggagaccacgctggtggccaaccagctccgcgaggcagcctccaggctcaagtggacaacaaacacaggccccacaccccaccaaactccgtaccagctggacccggccaacatcacgctggaacccatggaaatccgcactttcctggcctcagttcaatggaaggaggtggatggttaggtctgctgggatgggccctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccaccttagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaattcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 3 - DNA, 26 bp, Artificial Sequence
  oligonucleotide ICO1106
tctgcgctcgaggctgcctggactca

SEQ ID NO: 4 - DNA, 30 bp, Artificial Sequence
  oligonucleotide ICO1107
tgctaatctagagggcccatcccagcagac

SEQ ID NO: 5 - DNA, 20 bp, Artificial Sequence
  oligonucleotide ICO873
tgcagcttataatggttaca

SEQ ID NO: 6 - DNA, 20 bp, Artificial Sequence
  oligonucleotide ICO929
ggcacctattggtcttactg

SEQ ID NO: 7 - DNA, 20 bp, Artificial Sequence
  oligonucleotide ICO1110
aagtcgtgcgagaccttgtg

SEQ ID NO: 8 - DNA, 20 bp, Artificial Sequence
  oligonucleotide ICO1111
ccgccaaggaatctgtgctg

SEQ ID NO: 9 - DNA, 20 bp, Artificial Sequence
  oligonucleotide ICO1112
tacgagcgcctcagctacaa

SEQ ID NO: 10 - DNA, 20 bp, Artificial Sequence
  oligonucleotide ICO1113
gcggaaggtgaattggatgg

SEQ ID NO: 11 - DNA, 20 bp, Artificial Sequence
  oligonucleotide ICO1114
gcctcaggtgcctacatctt

SEQ ID NO: 12 - protein, 1011 aa, human
MGAYARASGVCARGCLDSAGPWTMSRALRPPLPPLCFFLLLLAAAGARAGGYETCPTVQPNMLNVHLLPHTHDDVGWLKTVDQYFYGIKNDIQHAGVQYILDSVISALLADPTRRFIYVEIAFFSRWWHQQTNATQEVVRDLVRQGRLEFANGGWVMNDEAATHYGAIVDQMTLGLRFLEDTFGNDGRPRVAWHIDPFGHSREQASLFAQMGFDGFFFGRLDYQDKWVRMQKLEMEQVWRASTSLKPPTADLFTGVLPNGYNPPRNLCWDVLCVDQPLVEDPRSPEYNAKELVDYFLNVATAQGRYYRTNHTVMTMGSDFQYENANMWFKNLDKLIRLVNAQQAKGSSVHVLYSTPACYLWELNKANLTWSVKHDDFFPYADGPHQFWTGYFSSRPALKRYERLSYNFLQVCNQLEALVGLAANVGPYGSGDSAPLNEAMAVLQHHDAVSGTSRQHVANDYARQLAAGWGPCEVLLSNALARLRGFKDHFTFCQQLNISICPLSQTAARFQVIVYNPLGRKVNWMVRLPVSEGVFVVKDPNGRTVPSDVVIFPSSDSQAHPPELLFSASLPALGFSTYSVAQVPRWKPQARAPQPIPRRSWSPALTIENEHIRATFDPDTGLLMEIMNMNQQLLLPVRQTFFWYNASIGDNESDQASGAYIFRPNQQKPLPVSRWAQIHLVKTPLVQEVHQNFSAWCSQVVRLYPGQRHLELEWSVGPIPVGDTWGKEVISRFDTPLETKGRFYTDSNGREILERRRDYRPTWKLNQTEPVAGNYYPVNTRIYITDGNMQLTVLTDRSQGGSSLRDGSLELMVHRRLLKDDGRGVSEPLMENGSGAWVRGRHLVLLDTAQAAAAGHRLLAEQEVLAPQVVLAPGGGAAYNLGAPPRTQFSGLRRDLPPSVHLLTLASWGPEMVLLRLEHQFAVGEDSGRNLSAPVTLNLRDLFSTFTITRLQETTLVANQLREAASRLKWTTNTGPTPHQTPYQLDPANITLEPMEIRTFLASVQWKEVDG